(19) United States
(12) Patent Application Publication
(10) Pub. No.: US 2020/0064322 A1
(43) Pub. Date: Feb. 27, 2020

(54) ELECTRONIC DEVICE FOR MEASURING GAS AND METHOD THEREFOR

(71) Applicant: SAMSUNG ELECTRONICS CO., LTD., Suwon-si, Gyeonggi-do (KR)

(72) Inventors: Hyun-Cheol PARK, Daegu (KR); Dong-Wook KIM, Seoul (KR); Sang II PARK, Gumi-si, Gyeongsangbuk-do (KR); Sung-Gun BAE, Hwaseong-si, Gyeonggi-do (KR); Ik-Joo BYUN, Suwon-si, Gyeonggi-do (KR); Tae-Han LEE, Seoul (KR); Tae-Han JEON, Hwaseong-si, Gyeonggi-do (KR); Tae-Ho KIM, Cheongju-si, Chungcheongbuk-do (KR); Jeong-Min PARK, Hwaseong-si, Gyeonggi-do (KR); Seung-Eun LEE, Seoul (KR)

(21) Appl. No.: 16/485,949

(22) PCT Filed: Feb. 1, 2018
(86) PCT No.: PCT/KR2018/001394

§ 371 Date: Aug. 14, 2019

(30) Foreign Application Priority Data
Feb. 16, 2017 (KR) 10-2017-0021180

(51) Int. Cl. G01N 33/00; G01K 13/00; G01N 27/12

(52) U.S. Cl. CPC G01N 33/0031; G01N 27/12; G01K 13/00; G01N 33/004

(57) ABSTRACT

An electronic device can comprise: a sensor module; and a processor for acquiring periphery state information indicating information on the outside of the electronic device and/or device state information indicating information on the inside of the electronic device, acquiring a measurement profile including information regarding target gas to be measured and a detection period of the target gas on the basis of the periphery state information and/or the device state information, and detecting the target gas through the sensor module according to the measurement profile.

### CROSS-REFERENCE TO RELATED APPLICATION(S)

This application is a National Phase Entry of PCT International Application No. PCT/KR2018/001394, which was filed on Feb. 1, 2018, and claims priority to Korean Patent Application No. 10-2017-0021180, which was filed on Feb. 16, 2017, the contents of which are incorporated herein by reference.

### BACKGROUND

### 1. Field

Various embodiments of the disclosure relate to an electronic device that measures gas and a processing method thereof.

### 2. Description of the Related Art

Gas sensors are elements that detect specific chemical substances contained in gas, convert concentrations thereof into an electrical signal, and output the electrical signal. Gas sensors vary according to sensing schemes. Typically, there are schemes such as a scheme using a change in solid physical properties by adsorption of gas or a reaction, a scheme using combustion heat, a scheme using an electrochemical reaction, and a scheme using physical properties.

With the advancement of gas sensor technology, gas sensors used in tabletop meters and air purifiers have been miniaturized to be applicable to mobile and portable devices. As gas sensors that may be used in mobile and portable devices, semiconductor gas sensors that utilize changes in solid properties are commonly used.

Semiconductor gas sensors include a metal oxide, an insulating layer below the metal oxide, and a hot wire. The semiconductor gas sensors may sense gas by adsorbing gas molecules on the surface of the metal oxide and using an electrical resistance that varies with adsorption.

### SUMMARY

Conventionally, a gas measurement device (for example, an air purifier or an air monitoring device) to which a gas sensor is applied is fixedly installed in one position. The fixed gas measurement device measures gas around an installed location and measures the quality of air containing the gas. Because of this immovability, the gas measurement device provides a user with gas measurement information of the location where the gas measurement device is installed, instead of gas measurement information around the user. Therefore, the user receives incorrect gas measurement information.

A conventional gas measurement device, once configured for gas sensing, operates according to a configuration. Particularly, a portable gas measurement device operates according to an initial configuration, despite a frequent change of the ambient environment. In this case, following problems arise.

For example, even when air is not polluted, the gas measurement device senses the gas at the maximum performance in the same manner as when the air is polluted. This leads to unnecessary operation of the gas measurement device and excessive power consumption. In addition, when a second gas needs to be measured due to the change of an ambient environment while a first gas is being measured, the gas measurement device continuously measures the first gas. This leads to the decline of gas selectivity of the gas measurement device. Therefore, a gas measurement device needs to be controlled to operate in accordance with an ambient environment or a device condition.

Various embodiments of the disclosure may provide an electronic device for collecting information relating to an environment surrounding the electronic device measuring gas and information relating to the operation of the electronic device, may acquire configuration information for gas measurement for the current situation from the collected information, and may control gas measurement on the basis of the acquired configuration information.

In addition, various embodiments of the disclosure may provide an electronic device for gas measurement wherein the configuration information is changed according to an ambient context or an electronic device state , and a gas sensor is adjusted based on the changed configuration information.

An electronic device according to various embodiments of the disclosure may include: a sensor module; and a processor configured to acquire at least one of ambient state information indicating information relating to the outside of the electronic device or device state information indicating information relating to the inside of the electronic device, acquire a measurement profile including information relating to a target gas to be measured and information relating to a sensing period of the target gas on the basis of at least one of the ambient state information and the device state information, and sense the target gas through the sensor module according to the measurement profile.

A control method of an electronic device according to various embodiments of the disclosure, which is a method of measuring gas by an electronic device including a sensor module, may include: acquiring at least one of ambient state information indicating information relating to the outside of the electronic device or device state information indicating information relating to the inside of the electronic device; acquiring a measurement profile including information relating to a target gas to be measured and information relating to a sensing period of the target gas on the basis of at least one of the ambient state information and the device state information; and sensing the target gas through the sensor module according to the measurement profile.

According to various embodiments of the disclosure, there may be provided an electronic device and a method thereof, by which a gas sensor can be controlled according to a profile optimized for an environment, so that accurate and useful gas measurement information can be provided to a user.

### BRIEF DESCRIPTION OF THE DRAWINGS

FIG. 1 illustrates a block diagram of an electronic device and a network according to various embodiments of the disclosure;

FIG. 2 is a block diagram of an electronic device according to various embodiments;

FIG. 3 is a block diagram of a program module according to various embodiments;

FIG. 4 is a block diagram of an electronic device according to various embodiments;

FIG. 5 illustrates a block diagram of at least one element of an electronic device according to an embodiment;

FIG. 6 illustrates a flow chart of a measurement profile configuration of an electronic device according to an embodiment;

FIG. 7 illustrates a conceptual diagram for explaining the relationship between a target gas and gas sensor temperature in an electronic device according to an embodiment;

FIG. 8 illustrates a flow chart of gas measurement by an electronic device according to an embodiment;

FIG. 9 illustrates a conceptual diagram for explaining a procedure of configuring a measurement profile according to various embodiments of the disclosure;

FIG. 10A illustrates conceptual diagrams for explaining an operation of performing output on a lock screen of an electronic device and an operation of performing output to another electronic device according to an embodiment;

FIG. 10B illustrates conceptual diagrams for explaining a user interface of an electronic device according to an embodiment;

FIG. 11 illustrates an array gas sensor according to various embodiments of the disclosure;

FIG. 12 illustrates a flow chart of gas measurement by an electronic device according to an embodiment;

FIG. 13 illustrates a flow chart of gas measurement by an electronic device according to an embodiment;

FIG. 14 illustrates a flow chart of repetitive sensing by an electronic device according to an embodiment;

FIG. 15 illustrates a flow chart of an operation of adjusting a sensing period on the basis of a change in ambient state information and device state information of an electronic device according to an embodiment;

FIG. 16 illustrates a flow chart of sensing period adjustment on the basis of a remaining amount of battery power of an electronic device according to an embodiment; and

FIG. 17 illustrates a flow chart of a processor operation in a standby mode according to an embodiment.

### DETAILED DESCRIPTION

Hereinafter, various embodiments of the disclosure will be described with reference to the accompanying drawings. The embodiments and the terms used herein are not intended to limit the technology disclosed herein to specific forms, and should be understood to include various modifications, equivalents, and/or alternatives to the corresponding embodiments. In describing the drawings, similar reference numerals may be used to designate similar constituent elements. A singular expression may include a plural expression unless they are definitely different in context. As used herein, the expression “A or B” or “at least one of A and/or B” may include all possible combinations of items enumerated together. The expression “a first”, “a second”, “the first”, or “the second” may modify various components regardless of the order and/or the importance, and is used merely to distinguish one element from any other element without limiting the corresponding elements. When an element (e.g., first element) is referred to as being “(functionally or communicatively) connected,” or “directly coupled” to another element (second element), the element may be connected directly to the another element or connected to the another element through yet another element (e.g., third element).

The expression “configured to” as used in various embodiments of the disclosure may be interchangeably used with, for example, “suitable for”, “having the capacity to”, “designed to”, “adapted to”, “made to”, or “capable of” in terms of hardware or software, according to circumstances. Alternatively, in some situations, the expression “device configured to” may mean that the device, together with other devices or components, “is able to”. For example, the phrase “processor adapted (or configured) to perform A, B, and C” may mean a dedicated processor (e.g., embedded processor) only for performing the corresponding operations or a generic-purpose processor (e.g., Central Processing Unit (CPU) or Application Processor (AP)) that may perform the corresponding operations by executing one or more software programs stored in a memory device.

An electronic device according to various embodiments of the disclosure may include at least one of, for example, a smart phone, a tablet Personal Computer (PC), a mobile phone, a video phone, an electronic book reader (e-book reader), a desktop PC, a laptop PC, a netbook computer, a workstation, a server, a Personal Digital Assistant (PDA), a Portable Multimedia Player (PMP), an MPEG-1 audio layer-3 (MP3) player, a mobile medical device, a camera, and a wearable device. According to various embodiments, the wearable device may include at least one of an accessory type (e.g., a watch, a ring, a bracelet, an anklet, a necklace, glasses, a contact lens, or a Head-Mounted Device (HMD)), a fabric or clothing integrated type (e.g., an electronic clothing), a body-mounted type (e.g., a skin pad, or tattoo), and a bio-implantable type (e.g., an implantable circuit). In some embodiments, the electronic device may include at least one of, for example, a television, a Digital Video Disk (DVD) player, a stereo, a refrigerator, an air conditioner, a vacuum cleaner, an oven, a microwave oven, a washing machine, an air cleaner, a set-top box, a home automation control panel, a security control panel, a TV box (e.g., Samsung HomeSync™, Apple TV™, or Google TV™), a game console (e.g., Xbox™ and PlayStation™), an electronic dictionary, an electronic key, a camcorder, and an electronic photo frame.

In other embodiments, the electronic device may include at least one of various medical devices (e.g., various portable medical measuring devices (a blood glucose monitoring device, a heart rate monitoring device, a blood pressure measuring device, a body temperature measuring device, etc.), a Magnetic Resonance Angiography (MRA), a Magnetic Resonance Imaging (MRI), a Computed Tomography (CT) machine, and an ultrasonic machine), a navigation device, a Global Positioning System (GPS) receiver, an Event Data Recorder (EDR) , a Flight Data Recorder (FDR), a Vehicle Infotainment Device, an electronic device for a ship (e.g., a navigation device for a ship, and a gyro-compass), avionics, security devices, an automotive head unit, a robot for home or industry, an Automatic Teller Machine (ATM) in banks, Point Of Sales (POS) in a shop, or an Internet of Things device (e.g., a light bulb, various sensors, electric or gas meter, a sprinkler device, a fire alarm, a thermostat, a street lamp, a toaster, a sporting goods, a hot water tank, a heater, a boiler, etc.). According to some embodiments, an electronic device may include at least one of a part of furniture or a building/structure, an electronic board, an electronic signature receiving device, a projector, and various types of measuring instruments (e.g., a water meter, an electric meter, a gas meter, a radio wave meter, and the like). In various embodiments, the electronic device may be flexible, or may be a combination of one or more of the aforementioned various devices. The electronic device according to embodiments of the disclosure is not limited to the above-described devices. In the disclosure, the term “user” may indicate a person using an electronic device or a device (e.g., an artificial intelligence electronic device) using an electronic device.

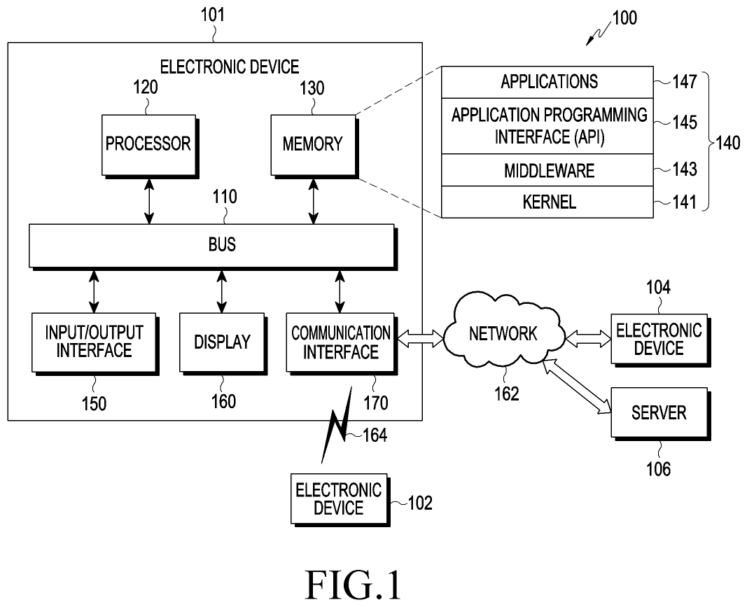

FIG. 1 illustrates a block diagram of an electronic device and a network according to various embodiments of the disclosure. Referring to FIG. 1, an electronic device 101 within a network environment 100 according to various embodiments will be described. The electronic device 101 may include a bus 110, a processor 120, a memory 130, an input/output interface 150, a display 160, and a communication interface 170. In some embodiments, the electronic device 101 may omit at least one of the elements, or may further include other elements. The bus 110 may include, for example, a circuit that interconnects the elements 110 to 170 and transfers communication (e.g., control messages or data) between the elements. The processor 120 may include one or more of a central processing unit, an application processor, and a communication processor (CP). The processor 120, for example, may carry out operations or data processing relating to the control and/or communication of at least one other element of the electronic device 101.

The memory 130 may include a volatile or non-volatile memory. The memory 130 may store, for example, instructions or data relevant to at least one other element of the electronic device 101. According to an embodiment, the memory 130 may store software and/or a program 140. The program 140 may include a kernel 141, middleware 143, an Application Programming Interface (API) 145, and/or application programs (or “applications”) 147. At least some of the kernel 141, the middleware 143, and the API 145 may be referred to as an operating system. The kernel 141 may control or manage system resources (for example, the bus 110, the processor 120, or the memory 130) used for executing an operation or function implemented by other programs (for example, the middleware 143, the API 145, or the application 147). Furthermore, the kernel 141 may provide an interface through which the middleware 143, the API 145, or the application programs 147 may access the individual elements of the electronic device 101 to control or manage the system resources.

The middleware 143 may function as, for example, an intermediary for allowing the API 145 or the application programs 147 to communicate with the kernel 141 to exchange data. Furthermore, the middleware 143 may process one or more task requests, which are received from the application programs 147, according to priorities thereof. For example, the middleware 143 may assign priorities for using the system resources (for example, the bus 110, the processor 120, the memory 130, or the like) of the electronic device 101 to one or more of the application programs 147, and may process the one or more task requests. The API 145 is an interface through which the applications 147 control functions provided from the kernel 141 or the middleware 143, and may include, for example, at least one interface or function (for example, instruction) for file control, window control, image processing, or text control. For example, the input/output interface 150 may forward instructions or data, input from a user or an external device, to the other element(s) of the electronic device 101, or may output instructions or data, received from the other element(s) of the electronic device 101, to the user or the another external device.

The display 160 may include, for example, a Liquid Crystal Display (LCD), a Light Emitting Diode (LED) display, an Organic Light Emitting Diode (OLED) display, a Micro Electro Mechanical System (MEMS) display, or an electronic paper display. The display 160 may display, for example, various types of contents (e.g., text, images, videos, icons, or symbols) to the user. The display 160 may include a touch screen and may receive, for example, a touch, gesture, proximity, or hovering input using an electronic pen or the user's body part. The communication interface 170 may establish, for example, communication between the electronic device 101 and an external device (for example, a first external electronic device 102, a second external electronic device 104, or a server 106). For example, the communication interface 170 may be connected to a network 162 through wireless or wired communication to communicate with the external device (for example, the second external electronic device 104 or the server 106).

The wireless communication may include, for example, a cellular communication that uses at least one of LTE, LTE-Advance (LTE-A), code division multiple access (CDMA), wideband CDMA (WCDMA), universal mobile telecommunications system (UMTS), wireless broadband (WiBro), global system for mobile communications (GSM), or the like. According to an embodiment, as illustrated in element 164 of FIG. 1, the wireless communication may include at least one among, for example, Wireless Fidelity (Wi-Fi), Light Fidelity (Li-Fi), Bluetooth, Bluetooth low power (BLE), ZigBee, near field communication (NFC) , magnetic secure transmission, a radio frequency (RF), or a body area network (BAN). According to an embodiment, the wireless communication may include a GNSS. The GNSS may be, for example, a global positioning system (GPS), a global navigation satellite system (Glonass), a Beidou navigation satellite system (Beidou) or Galileo, a European global satellite-based navigation system. Hereinafter, in this document, the term “GPS” may be interchangeable with the term “GNSS”. The wired communication may include, for example, at least one of a Universal Serial Bus (USB), a High Definition Multimedia Interface (HDMI), Recommended Standard 232 (RS-232), power line communication, a Plain Old Telephone Service (POTS), and the like. The network 162 may include a telecommunications network, for example, at least one of a computer network (for example, a LAN or a WAN), the Internet, and a telephone network.

Each of the first and second external electronic devices 102 and 104 may be of the same or a different type from the electronic device 101. According to various embodiments, all or some of the operations executed in the electronic device 101 may be executed in another electronic device or a plurality of electronic devices (for example, the electronic devices 102 and 104 or the server 106). According to an embodiment, when the electronic device 101 has to perform some functions or services automatically or in response to a request, the electronic device 101 may make a request for performing at least some functions relating thereto to another device (for example, the electronic device 102 or 104 or the server 106) instead of performing the functions or services by itself or in addition. Another electronic device (for example, the electronic device 102 or 104, or the server 106) may execute the requested functions or the additional functions, and may deliver a result thereof to the electronic device 101. The electronic device 101 may provide the received result as it is, or may additionally process the received result to provide the requested functions or services. To this end, for example, cloud computing, distributed computing, or client-server computing technology may be used.

In various embodiments of the disclosure, the processor may configure the target gas in the measurement profile, may configure a sensing temperature of the target, and may configure a sensing period of the target gas.

In various embodiments of the disclosure, the processor may configure, when the sensor module includes sensor cells that sense multiple gases, the target gas, the sensing temperature, and the sensing period for respective sensor cells in the measurement profile.

In various embodiments of the disclosure, the processor may configure the plurality of target gases in the measurement profile, and may configure sensing temperature and a sensing period for each of the multiple target gases.

In various embodiments of the disclosure, the processor may configure a sensing order or iterative sensing of the multiple target gases in the measurement profile.

In various embodiments of the disclosure, the processor may adjust the sensing period according to a measurement result of the target gas and reconfigure the sensing period to the adjusted sensing period.

In various embodiments of the disclosure, the processor may adjust the sensing period according to changes in the ambient state information and the device state information and may reconfigure the sensing period to the adjusted sensing period.

In various embodiments of the disclosure, the processor may adjust the sensing period according to submergence, sound data transmission or reception, a remaining battery amount of the electronic device, or an aspect in which the electronic device is kept, and may reconfigure the sensing period to the adjusted sensing period.

In various embodiments of the disclosure, the processor may adjust the sensing period according to a place where the electronic device is located and reconfigure the sensing period to the adjusted sensing period.

In various embodiments of the disclosure, the electronic device may further include: a display; and a communication module, wherein the processor may output a measurement result of the target gas to the display or may transmit the measurement result to another electronic device through the communication module.

In various embodiments of the disclosure, the processor may determine the sensing temperature on the basis of a type of target gas to be measured by the sensor module.

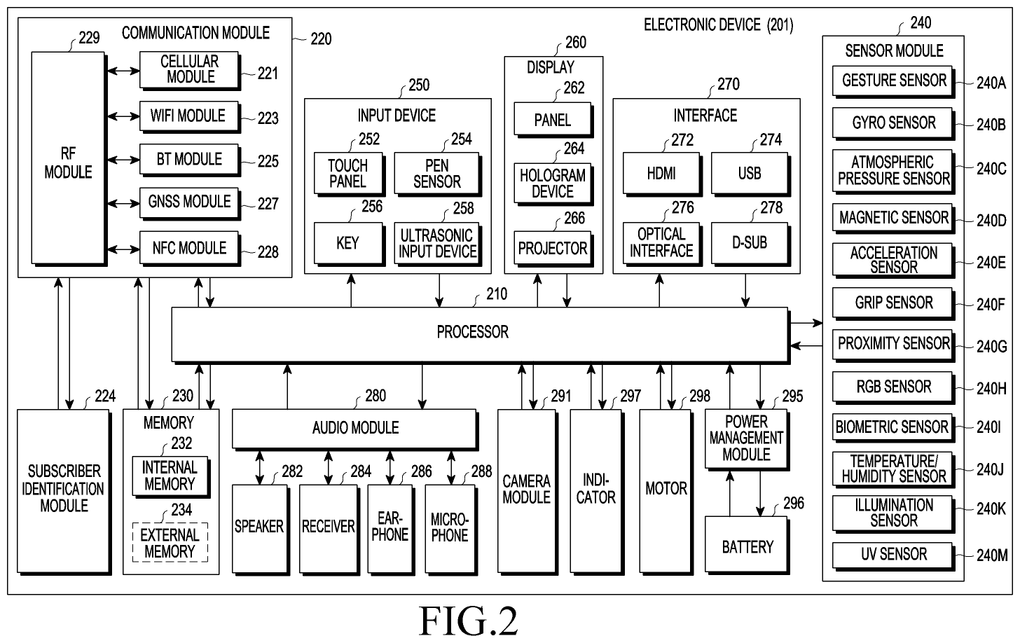

FIG. 2 is a block diagram of an electronic device 201 according to various embodiments. The electronic device 201 may include, for example, the whole or part of the electronic device 101 illustrated in FIG. 1. The electronic device 201 may include at least one processor 210 (e.g., an AP), a communication module 220, a subscriber identification module 224, a memory 230, a sensor module 240, an input device 250, a display 260, an interface 270, an audio module 280, a camera module 291, a power management module 295, a battery 296, an indicator 297, and a motor 298. The processor 210 may control a plurality of hardware or software elements connected thereto and may perform various data processing and operations by driving an operating system or an application program. The processor 210 may be implemented by, for example, a System on Chip (SoC). According to an embodiment, the processor 210 may further include a graphic processing unit (GPU) and/or an image signal processor. The processor 210 may also include at least some of the elements illustrated in FIG. 2 (e.g., a cellular module 221). The processor 210 may load, in volatile memory, instructions or data received from at least one of the other elements (for example, non-volatile memory), process the loaded instructions or data, and store the resultant data in the non-volatile memory.

The communication module 220 may have a configuration that is the same as, or similar to, that of the communication interface 170. The communication module 220 (for example, the communication interface 170) may include, for example, a cellular module 221, a Wi-Fi module 223, a Bluetooth module 225, a GNSS module 227, an NFC module 228, and an RF module 229. The cellular module 221 may provide, for example, a voice call, a video call, a text message service, an Internet service, or the like through a communication network. According to an embodiment, the cellular module 221 may identify and authenticate the electronic device 201 within a communication network using the subscriber identification module 224 (for example, a SIM card). According to an embodiment, the cellular module 221 may perform at least some of the functions that the processor 210 may provide. According to an embodiment, the cellular module 221 may include a communication processor (CP). In some embodiments, at least some (two or more) of the cellular module 221, the Wi-Fi module 223, the Bluetooth module 225, the GNSS module 227, and the NFC module 228 may be included in a single Integrated Chip (IC) or IC package. The RF module 229 may transmit/receive, for example, a communication signal (for example, an RF signal). The RF module 229 may include, for example, a transceiver, a power amp module (PAM), a frequency filter, a low noise amplifier (LNA), an antenna, or the like. According to another embodiment, at least one of the cellular module 221, the Wi-Fi module 223, the BT module 225, the GPS module 227, and the NFC module 228 may transmit/receive an RF signal through a separate RF module. The subscriber identification module 224 may include, for example, a card that includes a subscriber identity module and/or an embedded SIM, and may contain unique identification information (for example, an Integrated Circuit Card Identifier (ICCID)) or subscriber information (for example, an International Mobile Subscriber Identity (IMSI)).

The memory 230 (for example, the memory 130) may include, for example, an internal memory 232 or an external memory 234. The internal memory 232 may include, for example, at least one of a volatile memory (for example, a DRAM, an SRAM, an SDRAM, or the like) and a non-volatile memory (for example, a One Time Programmable ROM (OTPROM), a PROM, an EPROM, an EEPROM, a mask ROM, a flash ROM, a flash memory, a hard disc drive, or a Solid State Drive (SSD)). The external memory 234 may include a flash drive, for example, a compact flash (CF), a secure digital (SD), a Micro-SD, a Mini-SD, an eXtreme digital (xD), a multi-media card (MMC), a memory stick, and the like. The external memory 234 may be functionally and/or physically connected to the electronic device 201 through various interfaces.

The sensor module 240 may, for example, measure a physical quantity or detect the operating state of the electronic device 201 and may convert the measured or detected information into an electrical signal. The sensor module 240 may include, for example, at least one of a gesture sensor 240A, a gyro sensor 240B, an atmospheric pressure sensor 240C, a magnetic sensor 240D, an acceleration sensor 240E, a grip sensor 240F, a proximity sensor 240G, a color sensor 240H (for example, a red, green, blue (RGB) sensor), a biometric sensor 240I, a temperature/humidity sensor 240J, an illumination sensor 240K, a ultraviolet (UV) sensor 240M, or a gas sensor 240N. Additionally or alternatively, the sensor module 240 may include, for example, an e-nose sensor, an electromyography (EMG) sensor, an electroencephalogram (EEG) sensor, an electrocardiogram (ECG) sensor, an infrared (IR) sensor, an iris sensor, and/or a fingerprint sensor. The sensor module 240 may further include a control circuit for controlling one or more sensors included therein. In some embodiments, the electronic device 201 may further include a processor configured to control the sensor module 240 as a part of or separately from the processor 210, and may control the sensor module 240 while the processor 210 is in a sleep state. A sensor module 240N may sense a gas in the air. The gas sensor 240N may include at least one among a semiconductor sensor, a ceramic moisture sensor, a piezoelectric sensor, a contact combustion sensor, a solid electrolyte sensor, an electrochemical sensor, and an infrared absorption sensor. Further detailed description will be given later.

The input device 250 may include, for example, a touch panel 252, a (digital) pen sensor 254, a key 256, or an ultrasonic input device 258. The touch panel 252 may use, for example, at least one of a capacitive type, a resistive type, an infrared type, and an ultrasonic type. Furthermore, the touch panel 252 may further include a control circuit. The touch panel 252 may further include a tactile layer to provide a tactile reaction to a user. The (digital) pen sensor 254 may include, for example, a recognition sheet that is a part of, or separate from, the touch panel. The key 256 may include, for example, a physical button, an optical key, or a keypad. The ultrasonic input device 258 may detect ultrasonic waves, which are generated by an input tool, through a microphone (for example, a microphone 288) to identify data corresponding to the detected ultrasonic waves.

The display 260 (for example, the display 160) may include a panel 262, a hologram device 264, a projector 266, and/or a control circuit for controlling them. The panel 262 may be implemented to be, for example, flexible, transparent, or wearable. The panel 262, together with the touch panel 252, may be configured as one or more modules. According to an embodiment, the panel 262 may include a pressure sensor (or a POS sensor) which may measure a strength of pressure of a user's touch. The pressure sensor may be implemented so as to be integrated with the touch panel 252 or may be implemented as one or more sensors separate from the touch panel 252. The hologram device 264 may show a three dimensional image in the air by using an interference of light. The projector 266 may display an image by projecting light onto a screen. The screen may be located, for example, in the interior of, or on the exterior of, the electronic device 201. The interface 270 may include, for example, an HDMI 272, a USB 274, an optical interface 276, or a D-subminiature (D-sub) 278. The interface 270 may be included in, for example, the communication circuit 170 illustrated in FIG. 1. Additionally or alternatively, the interface 270 may, for example, include a mobile high-definition link (MHL) interface, a secure digital (SD) card/multi-media card (MMC) interface, or an infrared data association (IrDA) standard interface.

The audio module 280 may convert, for example, sound into an electrical signal, and vice versa. At least some elements of the audio module 280 may be included, for example, in the input/output interface 145 illustrated in FIG. 1. The audio module 280 may process sound information that is input or output through, for example, a speaker 282, a receiver 284, earphones 286, the microphone 288, and the like. The camera module 291 is a device that can photograph a still image and a moving image. According to an embodiment, the camera module 291 may include one or more image sensors (for example, a front sensor or a rear sensor), a lens, an image signal processor (ISP), or a flash (for example, an LED or xenon lamp). The power management module 295 may manage, for example, the power of the electronic device 201. According to an embodiment, the power management module 295 may include a power management integrated circuit (PMIC), a charger IC, or a battery or fuel gauge. The PMIC may use a wired and/or wireless charging method. Examples of the wireless charging method may include a magnetic resonance method, a magnetic induction method, an electromagnetic wave method, and the like. Additional circuits (for example, a coil loop, a resonance circuit, a rectifier, and the like) for wireless charging may be further included. The battery gauge may measure, for example, the residual amount of the battery 296 and a voltage, current, or temperature while charging. The battery 296 may include, for example, a rechargeable battery and/or a solar battery.

The indicator 297 may display a particular state, for example, a booting state, a message state, a charging state, or the like of the electronic device 201 or a part (for example, the processor 210) of the electronic device 201. The motor 298 may convert an electrical signal into a mechanical vibration and may generate a vibration, a haptic effect, or the like. The electronic device 201 may include a mobile TV support device (e.g., a CPU) that can process media data according to a standard, such as digital multimedia broadcasting (DMB), digital video broadcasting (DVB), mediaFlo™, and the like. Each of the above-described elements of hardware according to the disclosure may be configured with one or more components, and the names of the corresponding component elements may vary based on the type of electronic device. In various embodiments, an electronic device (for example, the electronic device 201) may omit some elements or may further include additional elements, or some of the elements of the electronic device may be combined with each other to configure one entity, in which case the electronic device may identically perform the functions of the corresponding elements prior to the combination.

The processor 210 according to various embodiments of the disclosure may acquire at least one of ambient state information indicating information relating to the outside of electronic device 101 or device state information indicating information relating to the inside of the electronic device 101, may acquire a measurement profile including information relating to a target gas to be measured and information relating to a sensing period of the target gas, on the basis of at least one of the ambient state information or the device state information, wherein the target gas is sensed through the sensor module 240 according to the measurement profile.

The processor 210 according to various embodiments of the disclosure may configure the target gas, may configure sensing temperature of the target gas, and may configure a sensing period of the target gas in the measurement profile.

The processor 210 according to various embodiments of the disclosure may configure, when the sensor module 240 includes sensor cells that sense multiple gases, the target gas, the sensing temperature, and the sensing period for each of the multiple cells in the measurement profile.

The processor 210 according to various embodiments of the disclosure may configure multiple target gases in the measurement profile, and may configure sensing temperature and a sensing period for each of the multiple target gases.

The processor 210 according to various embodiments of the disclosure may configure a sensing order or iterative sensing of the multiple target gases in the measurement profile.

The processor 210 according to various embodiments of the disclosure may adjust the sensing period according to measurement results of the target gases, and may reconfigure the sensing period to the adjusted sensing period.

The processor 210 according to the various embodiments of the disclosure may adjust the sensing period according to changes in the ambient state information and the device state information and may reconfigure the sensing period to the adjusted sensing period.

The processor 210 according to the various embodiments of the disclosure may adjust the sensing period according to submergence, sound data transmission or reception, a remaining battery amount of the electronic device, or an aspect in which the electronic device is kept, and may reconfigure the sensing period to the adjusted sensing period.

The processor 210 according to various embodiments of the disclosure may adjust the sensing period according to a place where the electronic device 101 is located, and may reconfigure the sensing period to the adjusted sensing period.

The processor 210 according to various embodiments of the disclosure may output measurement results of the target gases to the display 260, or may transmit the measurement results to another electronic device through the communication module 220.

The processor 210 according to various embodiments of the disclosure may determine the sensing temperature on the basis of a type of the target gas to be measured by the sensor module 240.

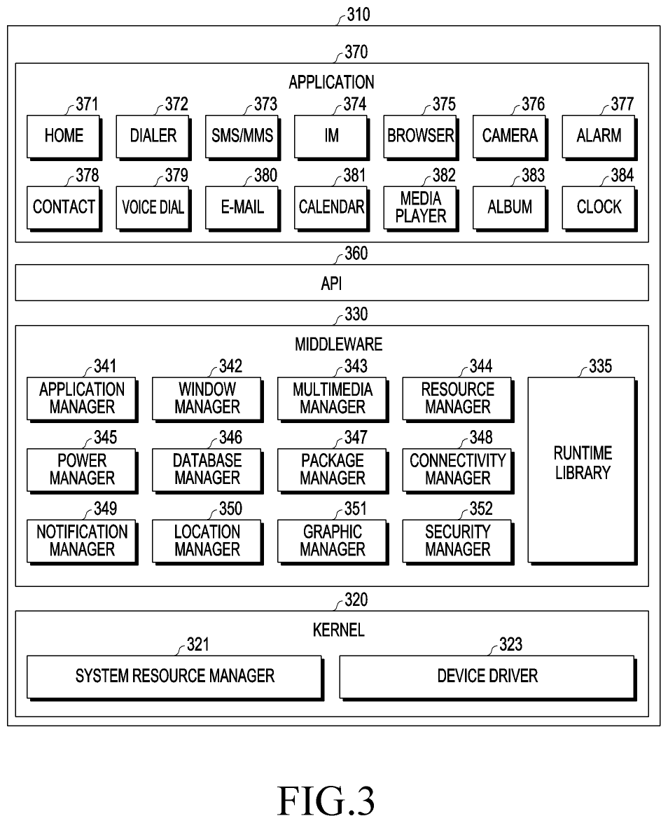

FIG. 3 is a block diagram of a program module according to various embodiments. According to an embodiment, the program module 310 (for example, the program 140) may include an Operating System (OS) that controls resources relating to an electronic device (for example, the electronic device 101) and/or various applications (for example, the application programs 147) that are driven on the operating system. The operating system may include, for example, Android™, iOS™, Windows™, Symbian™, Tizen™, or Bada™. Referring to FIG. 3, the program module 310 may include a kernel 320 (for example, the kernel 141), middleware 330 (for example, the middleware 143), an API 360 (for example, the API 145), and/or applications 370 (e.g., the application programs 147). At least a part of the program module 310 may be preloaded on the electronic device, or may be downloaded from an external electronic device (for example, the electronic device 102 or 104 or the server 106).

The kernel 320 may include, for example, a system resource manager 321 and/or a device driver 323. The system resource manager 321 may control, allocate, or retrieve system resources. According to an embodiment, the system resource manager 321 may include a process manager, a memory manager, or a file system manager. The device driver 323 may include, for example, a display driver, a camera driver, a Bluetooth driver, a shared memory driver, a USB driver, a keypad driver, a Wi-Fi driver, an audio driver, or an inter-process communication (IPC) driver. The middleware 330 may provide, for example, a function required by the applications 370 in common, or may provide various functions to the applications 370 through the API 360 such that the applications 370 can efficiently use limited system resources within the electronic device. According to an embodiment, the middleware 330 may include at least one of a runtime library 335, an application manager 341, a window manager 342, a multi-media manager 343, a resource manager 344, a power manager 345, a database manager 346, a package manager 347, a connectivity manager 348, a notification manager 349, a location manager 350, a graphic manager 351, and a security manager 352.

The runtime library 335 may include, for example, a library module that a compiler uses in order to add a new function through a programming language while the applications 370 are being executed. The runtime library 335 may manage an input/output, manage a memory, or process an arithmetic function. The application manager 341 may manage, for example, the life cycles of the applications 370. The window manager 342 may manage GUI resources used for a screen. The multimedia manager 343 may identify formats required for reproducing various media files and may encode or decode a media file using a codec suitable for the corresponding format. The resource manager 344 may manage the source code of the applications 370 or the space in memory. The power manager 345 may manage, for example, the capacity or power of a battery and may provide power information required for operating the electronic device. According to an embodiment, the power manager 345 may operate in conjunction with a basic input/output system (BIOS). The database manager 346 may, for example, generate, search, or change databases to be used by the applications 370. The package manager 347 may manage the installation or update of an application that is distributed in the form of a package file.

The connectivity manager 348 may manage, for example, a wireless connection. The notification manager 349 may provide information on an event (for example, an arrival message, an appointment, a proximity notification, or the like) to a user. The location manager 350 may manage, for example, the location information of the electronic device. The graphic manager 351 may manage a graphic effect to be provided to a user and a user interface relating to the graphic effect. The security manager 352 may provide, for example, system security or user authentication. According to an embodiment, the middleware 330 may include a telephony manager for managing a voice or video call function of the electronic device or a middleware module that is capable of forming a combination of the functions of the above-described elements. According to an embodiment, a middleware 330 may provide a module specialized for each type of operating system. Furthermore, the middleware 330 may dynamically remove some of the existing elements, or may add new elements. The API 360 is, for example, a set of API programming functions, and may be provided with different configurations depending on the operating system. For example, in the case of Android or iOS, one API set may be provided for each platform, and in the case of Tizen, two or more API sets may be provided for each platform.

An application 370 may include home 371, a dialer 372, an SMS/MMS 373, an instant message 374, a browser 375, a camera 376, an alarm 377, a contact 378, a voice dial 379, email 380, a calendar 381, a media player 382, an album 383, a watch 384, healthcare (e.g., measurement of the amount of exercise or blood glucose) or environmental information (e.g., atmospheric pressure, humidity, or temperature information) providing application. According to an embodiment, the applications 370 may include an information exchange application that can support the exchange of information between the electronic device and an external electronic device. The information exchange application may include, for example, a notification relay application for relaying particular information to an external electronic device or a device management application for managing an external electronic device. For example, the notification relay application may relay notification information generated in the other applications of the electronic device to an external electronic device, or may receive notification information from an external electronic device to provide the received notification information to a user. A device management application may install, delete, or update a function (e.g., turning on/turning off of an external device itself (or some elements) or adjustment of brightness (or resolution) of the display) of an external device that communicates with the electronic device or an application operating in the external electronic device. According to an embodiment, the applications 370 may include applications (for example, a health care application of a mobile medical appliance) that are designated according to the attributes of an external electronic device. According to an embodiment, the applications 370 may include applications received from an external electronic device. At least some of the program module 310 may be implemented (for example, executed) by software, firmware, hardware (for example, the processor 210), or a combination of two or more thereof and may include a module, a program, a routine, an instruction set, or a process for performing one or more functions.

The term “module” as used herein may include a unit consisting of hardware, software, or firmware, and may, for example, be used interchangeably with the term “logic”, “logical block”, “component”, “circuit”, or the like. The “module” may be an integrated component, or a minimum unit for performing one or more functions or a part thereof The “module” may be mechanically or electronically implemented and may include, for example, an Application-Specific Integrated Circuit (ASIC) chip, a Field-Programmable Gate Arrays (FPGA), or a programmable-logic device, which has been known or are to be developed in the future, for performing certain operations. At least some of devices (e.g., modules or functions thereof) or methods (e.g., operations) according to various embodiments may be implemented by an instruction which is stored a computer-readable storage medium (e.g., the memory 130) in the form of a program module. The instruction, when executed by a processor (e.g., the processor 120), may cause the one or more processors to execute the function corresponding to the instruction. The computer-readable storage medium may include a hard disk, a floppy disk, a magnetic medium (e.g., a magnetic tape), an Optical Media (e.g., CD-ROM, DVD), a Magneto-Optical Media (e.g., a floptical disk), an inner memory, etc. The instruction may include a code made by a compiler or a code that can be executed by an interpreter. The programming module according to the disclosure may include one or more of the aforementioned components or may further include other additional components, or some of the aforementioned components may be omitted. Operations performed by a module, a programming module, or other elements according to various embodiments may be executed sequentially, in parallel, repeatedly, or in a heuristic manner. At least some operations may be executed according to another sequence, may be omitted, or may further include other operations.

The electronic device 101 according to various embodiments of the disclosure may determine various states during a call, in addition to whether the call is made indoors or in a vehicle. For example, the electronic device 101 may determine whether the electronic device 101 is located in a bag. The electronic device 101 may determine that the electronic device 101 is located in a bag if it is determined that data sensed by the illumination sensor shows an environment in which light is blocked. In this case, the electronic device 101 may configure a measurement period to be relatively long. The electronic device 101 according to various embodiments of the disclosure may determine whether the electronic device 101 has been submerged. The electronic device 101 may determine whether submergence has occurred, on the basis of a measurement result of various submergence determination circuits included therein. When the electronic device 101 is determined to be submerged, the electronic device 101 may stop measurement.

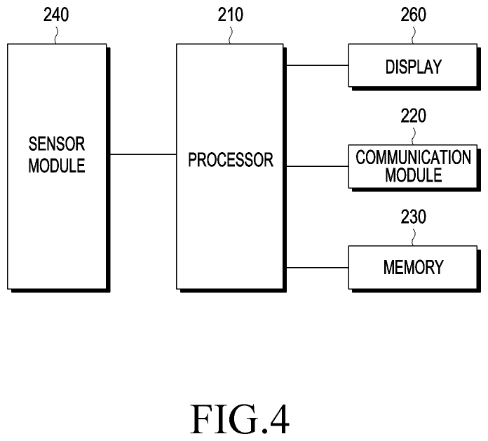

FIG. 4 illustrates a block diagram of the electronic device according to an embodiment.

Referring to FIG. 4, the electronic device 101 may include the sensor module 240, the processor 210, the display 260, the communication module 220, and the memory 230.

The sensor module 240 may collect data for information acquisition by contacting or being close to an object to be measured. The sensor module 240 may be controlled by the processor 210. The processor 210 may control the sensor module 240 to collect data and transmit the collected data to the processor 210.

In various embodiments of the disclosure, the sensor module 240 may be implemented as a sensor hub. In this case, the sensor hub may not only collect data for information acquisition by contacting or being close to an object to be measured, but also temporarily store the data. The sensor hub may store the data and then transmit the data to the processor 210. The sensor hub may be controlled by the processor 210, or may control itself to acquire, store, manage, and transmit data. In this case, the sensor hub may further include, inside thereof, a processor capable of performing calculation. The sensor hub may be controlled to collect data, store the data, and transmit the data to the processor 210. Therefore, even when the processor 210 is in a sleep state, the sensor hub may continuously collect and store data, and when the processor 210 wakes up, the sensor hub may transfer the collected data to the processor 210 accordingly.

When the sensor hub receives a data transmission request from the processor 210, the sensor hub may transmit data to the processor 210. The sensor hub may store the data until the sensor hub receives the data transmission request.

The sensor module 240 may collect data for acquiring ambient state information and device state information.

The ambient state information may include information relating to the outside of the electronic device 101. An ambient state may refer to an environment surrounding the electronic device 101. The ambient state may include a user carrying the electronic device 101 and an environment surrounding the user. Therefore, the ambient state information may be divided into state information of the user and state information of the environment surrounding the user.

The user state information may include health information of the user. For example, the user state information may include at least one among a magnitude of stress received by the user, chronic or acute disease, fatigue, and drowsiness. The user state information may include movement information of the user. For example, the user state information may relate to whether the user is in motion or is traveling.

The environment state information may include information of a place where the user is located. For example, the environment state information may relate to whether the user is indoors or outdoors and whether the user is in a vehicle. The environment state information may include time information. For example, the time information may include current time information. The environment state information may include weather information. For example, the weather information may include at least one among temperature, precipitation, humidity, wind speed, and the like. The environment state information may include area information. For example, the area information may relate to whether the area in which the user is located is in a city with a high air pollution degree or near the city with a low air pollution degree.

The device state information may include information relating to the inside of the electronic device 101. A device state may refer to operation or characteristics of the electronic device 101. Therefore, the device state information may be divided into state information of operation and state information of characteristics of the electronic device 101.

For example, the operation state information may relate to whether the electronic device 101 is being charged, receiving a touch input, transmitting or receiving data, being submerged (contacting with moisture), or being stored in a bag or pocket.

The characteristic state information may relate to a currently executed application or the remaining battery amount.

The processor 210 may receive data. The processor 210 may receive collected data that is collected by the sensor module 240. The collected data may include all data collected by the sensor module 240. In particular, the processor 210 may receive data for ambient state information and device state information. The processor 210 may receive the collected data from the sensor module 240.

The processor 210 may process data. In particular, the processor 210 may process the collected data. The processor 210 may process the collected data so as to acquire ambient state information and device state information.

The processor 210 may control the sensor module 240. Preferably, the processor 210 may configure a measurement profile and may operate the sensor module 240 according to the configured measurement profile. The sensor module 240 may sense gas according to operation of the processor 210. The measurement profile may include, as a condition for a kind of gas measurement, how the sensor module 240 should operate.

In configuration of the measurement profile, the processor 210 may configure items constituting the measurement profile from the peripheral state information or the device state information. The processor 210 may determine at least one of the ambient state information and the device state information, and may directly determine and configure items of the measurement profile. For configuration of the measurement profile, the processor 210 may use pre-stored data as it is. In particular, the processor 210 may read data matching one of the ambient state information and the device state information from the memory 230, and may configure items of the measurement profile on the basis of the read data.

The processor 210 may process gas sensing data in order to output the gas sensing data including a result of sensing gas. The processor 210 may control the display 260 to output the gas sensing data itself or the processed gas sensing data.

The processor 210 may include a user interface (UI) to output the gas sensing data. The user interface may be variously implemented by at least one of visual, auditory, and tactile methods to provide the gas sensing data to a user.

The processor 210 may process the gas sensing data to store the gas sensing data. The processor 210 may control the memory 230 to store the gas sensing data itself or the processed gas sensing data.

The processor 210 may process the gas sensing data to transmit the gas sensing data to another electronic device 101. The processor 210 may control the communication module 220 to transmit the gas sensing data itself or the processed gas sensing data to the another electronic device 101.

The display 260 may output data. The output of the display 260 may be implemented by at least one of visual, auditory, and tactile methods. The display 260 may output the gas sensing data via sound. The display 260 may output the gas sensing data via an image or a text message. The display 260 may output the gas sensing data and alert the user through vibration. The display 260 may display the gas sensing data together with an application that is being executed or on a screen (e.g., a lock screen).

The communication module 220 may transmit or receive data. The communication module 450 may receive data for acquiring the ambient state information or the device state information. The communication module 220 may transmit the gas sensing data or the gas sensing data processed by the processor 210 to the another electronic device 101. The communication module 220 may use a wired network or a wireless network. Preferably, the communication module 220 may operate on the basis of a wireless communication technology using a wireless communication network. The wireless communication technology may include Wireless Fidelity (Wi-Fi), Bluetooth, mobile communication, and Near Field Communication (NFC).

The memory 230 may store data. The memory 230 may store the collected data, the gas sensing data, and data obtained by processing both of the data. Further, the memory 230 may store measurement profile data and control command data for controlling another module by the processor 210. Here, the processed collected data and the measurement profile data may be matched with each other and stored.

Suppose, for example, that the processed collected data including the ambient state information and the device state information represents a motion mode. The motion mode may indicate a situation in which the electronic device 101 is in a pocket of a user currently in motion. The electronic device 101 may read, from the memory 230, measurement profile data corresponding to the motion mode, and may configure an item of the measurement profile according to the read measurement profile data. The electronic device 101 may sense gas by operating the sensor module 240 according to the configuration of the motion measurement profile. It is preferable that the measurement profile data and the processed collected data for the motion mode are stored together in the memory 230.

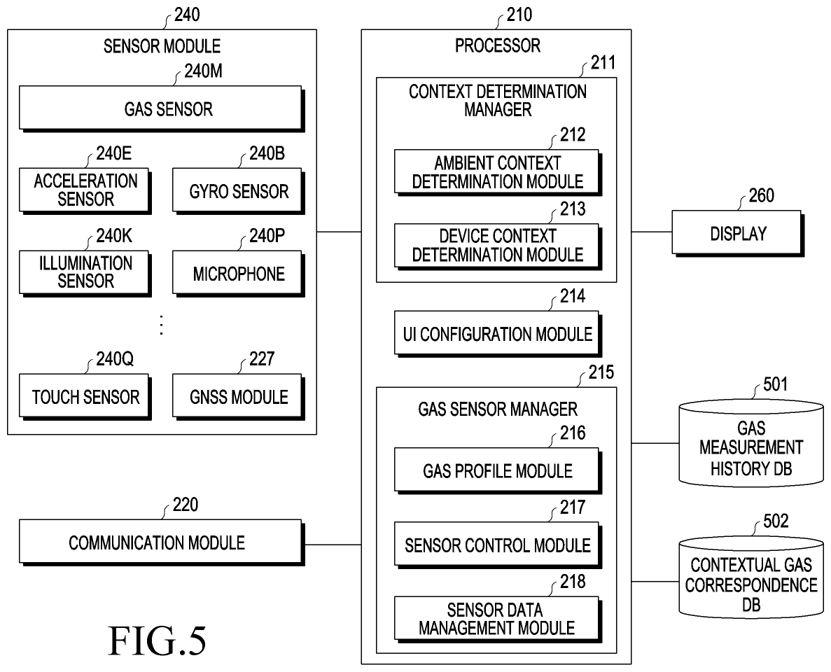

FIG. 5 illustrates a block diagram of at least one element of the electronic device according to an embodiment.

The sensor module 240 may include the gyro sensor 240B, the gas sensor 240N, the acceleration sensor 240E, the illumination sensor 240K, the microphone 240P, the touch sensor 204Q, and the GNSS module 227. The GNSS module 227 may be included in the sensor module 240 depending on implementation. Although not illustrated, the sensor module 240 may further include sensors, such as an ultrasonic sensor, a Heart Rate Monitoring (HRM) sensor, an electrocardiogram (ECG) sensor, an electroencephalography (EEG) sensor, an ElectroOculoGraphy (EOG) sensor, and an Electromyogram (EMG) sensor, which can measure a surrounding environment, a state and location of a user, and a context of the electronic device. The GNSS module 227 may acquire GPS information for determination of location information of a user. The gas sensor 240 N may include a sensor capable of measuring a gas level, for example, a gas concentration, etc., responsively to the gas.

In various embodiments of the disclosure, the communication module 220 may include a Wi-Fi communication module for transmitting and receiving a Wi-fi signal used to deduce a specific location, a Bluetooth module capable of transmitting measured gas-related information, or the like.

In various embodiments of the disclosure, the processor 210 may include at least one among a context determination manager 211, a UI configuration module 214, or a gas sensor manager 215. The context determination manager 211 may include at least one among a ambient context determination module 212 or a device context determination module 213. The ambient context determination module 212 may determine an ambient environment, a user state and location, and the like. The ambient context determination module 212 may predict a user health condition (e.g., a stress level, drowsiness, chronic disease, etc.) and user environment information (e.g., vehicle, office, home, park, during exercise, etc.) through information sensed by the sensor module 240. For example, the ambient context determination module 212 may determine that the user is driving in a car and is in a drowsy state, or the user is on the way to work on foot at 8:00 a.m. and the weather is very cold.

The device context determination module 213 may determine information related to an application executed in the background, charging-related information, battery information, information on whether a call is in progress, information on whether a user touch is detected, information relating to submergence, and information on whether the device is inside a bag or a pocket. For example, the device context determination module 213 may determine that the remaining battery amount is 30% or less and gas measurement has been last performed 10 minutes ago, while determining that a user is outdoors at 2:00 p.m.

The UI configuration module 214 may include a UI displayed on the display 260, and may include, for example, a UI including information determined in relation to gas.

The gas sensor manager 215 may include at least one of a gas profile module 216, a sensor control module 217, or a sensor data management module 218. The gas profile module 216 may determine at least one target gas, and may determine a period for measuring the determined target gas, that is, a measurement period. In various embodiments of the disclosure, the gas profile module 216 may configure at least one target gas by using user context and state information and device context information. The gas profile module 216 may determine the target gas by using information from a contextual gas correspondence database 502. The contextual gas correspondence database 502 may store various contexts and associated information of a target gas corresponding thereto, and the gas profile module 216 may configure the target gas by using the associated information. For example, the gas profile module 216 may configure a target gas for a room environment, such as home and office, where there are few people but a lot of furniture and facilities, and may configure a measurement profile, such as a measurement period in the room environment. The gas profile module 216 may configure, as a target gas, a gas generated from a human body in a place, such as a vehicle or a café, where contamination is mainly generated by a person, and may configure a measurement profile, such as a measurement period corresponding thereto. Accordingly, the gas sensor 240N may measure odor generated in the mouth of a person, by using the configured target gas or measurement profile. The gas profile module 216 may configure a gas measurement period by using user context and device state information. For example, the gas profile module 216 may configure a measurement period corresponding to the user context and device context by referring to information from a gas measurement history database 501. For example, the gas profile module 216 may determine a measurement period such that gas is measured more frequently, by configuring a measurement period to be short when it is determined that high concentration gas is exposed or a charging is progressed. Alternatively, the gas profile module 216 may determine a measurement period such that gas is measured less frequently, by configuring a measurement period to be long when it is determined that the device is submerged or is inside a pocket or a bag.

In various embodiments of the disclosure, the sensor control module 217 may perform sensor control corresponding to a form of a sensor. For example, when the sensor is a metal oxide sensor, the sensor control module 217 may perform sensor control by controlling a sensor temperature and the amount of current which correspond to a target gas. For example, when the sensor is an array form, the sensor control module 217 may perform sensor control by controlling a sensor operation specific to an individual target gas or a sensor operation specific to each array to be performed differently according to a corresponding state.

A sensor data measurement module 218 may process measured gas information as information to be provided to a user, and may update information to a database (e.g., 501 or 502).

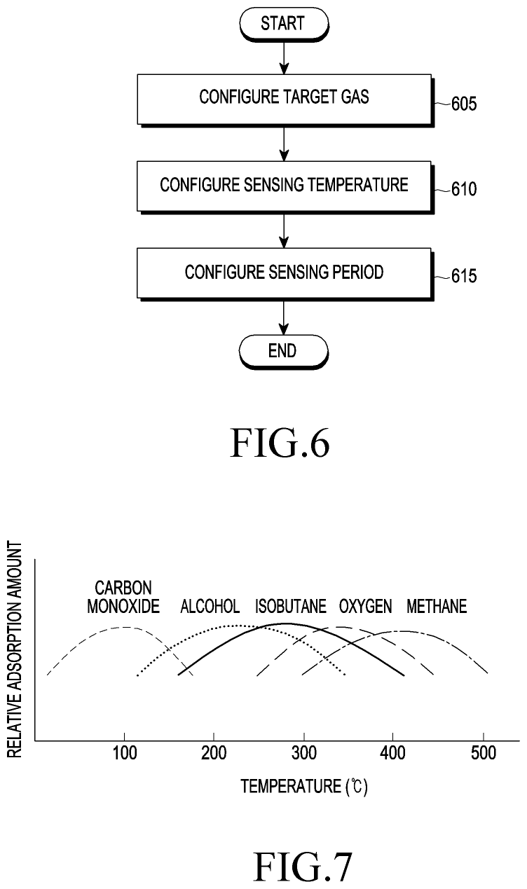

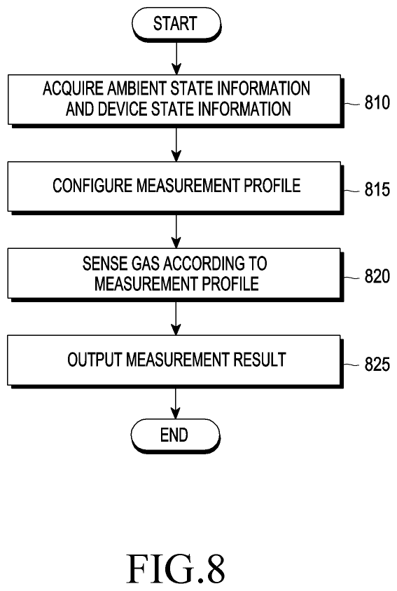

FIG. 6 illustrates a flow chart of a measurement profile configuration of an electronic device according to an embodiment. The embodiment of FIG. 6 will be described with reference to FIG. 7. FIG. 7 illustrates a conceptual diagram for explaining the relationship between a target gas and gas sensor temperature in the electronic device according to an embodiment.

Referring to FIG. 6, the measurement profile configuration of electronic device 101 may include a target gas configuration, a sensing temperature configuration, and a sensing period configuration.

In operation 605, the electronic device 101 or the processor 210 may configure a target gas on the basis of at least one of ambient state information or device state information. For example, when it is determined that the electronic device 101 is located indoors according to the ambient state information, the electronic device 101 may configure, as the target gas, at least one of oxygen and carbon dioxide affecting indoor air quality. Associated information with the target gas and at least one of the ambient state information and the device state information may be pre-stored in the electronic device 101. Accordingly, the electronic device 101 may configure or determine the target gas corresponding to at least one of the ambient state information or the device state information by using the pre-stored associated information.

In operation 610, the electronic device 101 may configure a sensing temperature on the basis of at least one of the ambient state information or the device state information. The sensing temperature may include temperature of the gas sensor 240N for sensing a specific target gas. For example, when the gas sensor 240N is a semiconductor sensor, the sensing temperature may include temperature of the surface of the gas sensor 240N contacting the target gas. Alternatively, the electronic device 101 may determine temperature corresponding to the target gas. For example, the electronic device 101 may pre-store associated information relating to a sensing temperature specific to each target gas, and may configure a sensing temperature corresponding to the configured target gas by using the associated information.

For example, referring to FIG. 7, a temperature range of the gas sensor 240N for determining an adsorption amount (sensing amount) according to types of gases is illustrated. The gas sensor 240N may sense different gases with relatively high adsorption amounts according to temperatures. Temperature values of the highest adsorption amounts of the gas sensor 240N according to types of gases are illustrated. The gas sensor 240N may adjust a degree of precision of adsorption (sensing) of any one gas according to temperature.

For example, the temperature at which carbon monoxide is best sensed is 100° C. and the temperature range in which carbon monoxide is sensed is ±80° C. based on 100° C. Therefore, the gas sensor 240N may sense carbon monoxide best at 100° C. At 180° C., the gas sensor 240N may sense not only carbon monoxide but also alcohol and isobutane. Therefore, it may not be efficient to configure a sensing temperature of the gas sensor 240N to be 180° C. to sense carbon monoxide. However, when carbon monoxide, isobutane, and alcohol need to be sensed together, it may be reasonable to configure the sensing temperature of the gas sensor 240N to be 180° C.

In order for oxygen to be sensed best among the target gases, the sensing temperature of 350° C. may be most appropriate. Therefore, the electronic device 101 may configure the sensing temperature of the gas sensor 240N to be 350° C.

In operation 615, the electronic device 101 may configure a sensing period on the basis of at least one of ambient state information or device state information. The sensing period may include the number of times of sensing the target gas by the gas sensor 240N according to a time unit. For example, when it is determined that an air quality is important or a user's health becomes worse, on the basis of the ambient state information, the electronic device 101 may perform gas sensing relatively frequently by decreasing the sensing period.

When it is determined that the electronic device 101 is located indoors or in a closed space, such as inside a vehicle, on the basis of the ambient state information, due to a high possibility of deterioration of the air quality, the electronic device 101 may perform gas sensing relatively more frequently. When it is determined that the electronic device 101 is located outdoors on the basis of the ambient state information, due to a low possibility of deterioration of the air quality, the electronic device 101 may perform gas sensing relatively less frequently by increasing the sensing period.

As described, the electronic device 101 may individually determine each of the target gas, the sensing period, and the like on the basis of at least one of the ambient state information and the device state information. However, in order to determine an element of a measurement profile, such as the target gas, the sensing temperature, and the sensing period, the electronic device 101 may read data relating to the element of the measurement profile from the memory 230 and refer the read data. For example, when the electronic device 101 determines the target gas, the electronic device 101 may read data relating to a configuration of a sensing temperature, a sensing period, or a type of a gas sensor, corresponding to the determined target gas.

In various embodiments of the disclosure, the electronic device 101 may include multiple gas sensors to measure specific gases, respectively. In this case, the electronic device 101 may select a gas sensor corresponding to the target gas from among the multiple gas sensors, and may measure the target gas by using the selected gas sensor.

In various embodiments of the disclosure, a sensing period, a sensing temperature, or a type of the gas sensor may be predetermined according to the target gas and stored in the memory 230. For example, when the electronic device 101 determines oxygen as the target gas, the electronic device 101 may read, from the memory 230, data in which the sensing temperature is 350° C., the sensing period is 30 times/sec, and the type of the gas sensor is a semiconductor gas sensor. Based on the read data, the electronic device 101 may activate the semiconductor gas sensor, and may configure the sensing temperature of the semiconductor gas sensor to be 350° C. and the sensing period to be 30 times/sec. Therefore, the gas sensor 240N senses oxygen 30 times per second.

In various embodiments of the disclosure, the electronic device 101 may determine a sensing order. For example, the electronic device 101 may determine multiple target gases, and may determine a sensing order according to priorities of the respective multiple target gases. In another embodiment, if the electronic device 101 includes multiple gas sensors, the electronic device 101 may concurrently measure the multiple target gases.

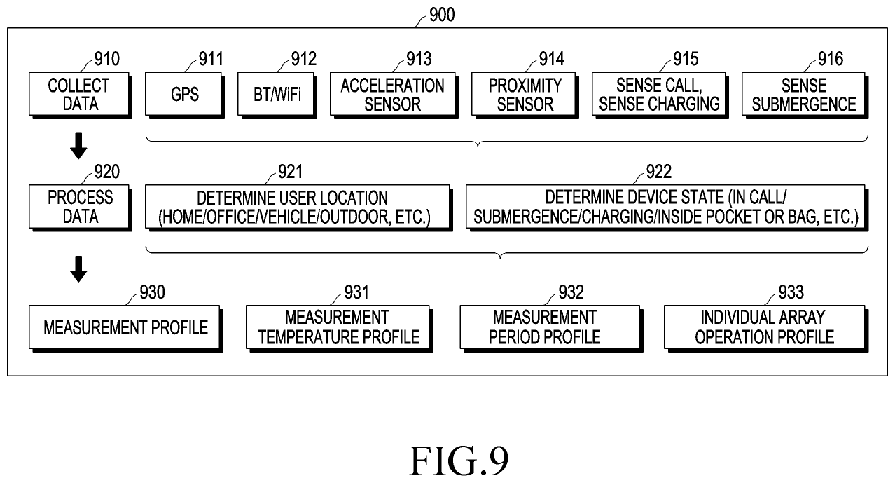

FIG. 8 illustrates a flow chart of gas measurement by the electronic device according to an embodiment.

In operation 810, the electronic device 101 or the processor 210 may acquire ambient state information and device state information by processing the collected data. The electronic device 101 may acquire at least one of the ambient state information or the device state information. The electronic device 101 may determine that a place where the electronic device 101 is located is an indoor space, by collecting data relating to movement, weather, and illumination, and may acquire ambient context information relating thereto. The electronic device 101 may determine that a user is on the phone, by sensing heat emitted from the user, transmission/reception of sound data, and a touch of the user, and may acquire device state information relating thereto.

In operation 815, the electronic device 101 may configure a measurement profile including a target gas required to be measured and information relating to a measurement condition of the target gas, on the basis of at least one of the acquired ambient state information or device state information. For example, the processor 210 may read, from the memory 230, data for measurement profile configuration, and may configure the measurement profile by using the read data.

The target gas may be an object to be monitored by the electronic device 101. When gas measurement is performed, what the electronic device 101 actually measures may be air containing multiple gases instead of a single gas. Therefore, the target gas may include not only one type of gas but also many types of gases. For example, the target gas in an indoor space may include oxygen, carbon dioxide, nitrogen, or the like. This is because oxygen, carbon dioxide, or nitrogen affect smooth breathing of a user in the indoor space. The target gas when the user is on the phone may include Volatile Sulfur Compounds (VSC). This is because the VSC may be a main cause of bad breath.

The measurement condition, that is the measurement profile, may serve as a kind of guideline for sensing the target gas by the electronic device 101. The measurement profile may include what gas the gas sensor 240N senses as the target gas, how the gas sensor 240N senses the target gas, and what the specifications and operations are (e.g., temperature of a gas adsorption surface, a frequency, and a gas sensor type) of the gas sensor 240N.

In operation 820, the electronic device 101 may sense the target gas according to a condition configured in the measurement profile. For example, the electronic device 101 may sense a gas including oxygen, carbon dioxide, nitrogen, or the like in the indoor space. If the user is on the phone, the electronic device 101 may sense a gas containing the volatile sulfur compound, which is a major component of bad breath.

In operation 825, the electronic device 101 may output a result of gas sensing as a measurement result. In particular, the electronic device 101 may process the sensed gas data to acquire measurement result information, and may output the measurement result information.

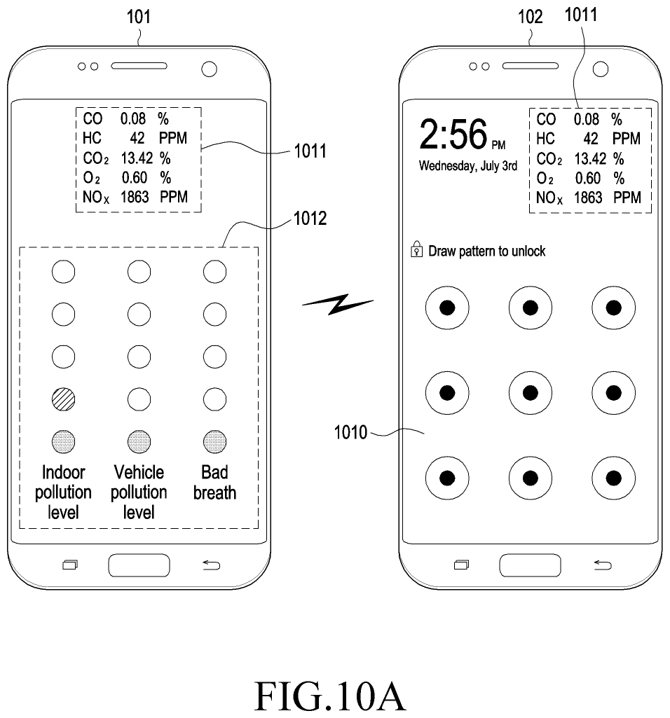

FIG. 9 illustrates a conceptual diagram for explaining a procedure of configuring a measurement profile according to various embodiments of the disclosure.

In operation 910, the electronic device 101 may collect data (DATA). The electronic device 101 according to various embodiments of the disclosure may collect various data, such as GPS information 911, Bluetooth/Wi-Fi (BT/Wi-Fi) information 912, acceleration sensor information 913, proximity sensor information 914, call sensing or charging sensing information 915, submergence sensing information 916, or the like.

In operation 920, the electronic device 101 may process the collected data, For example, the electronic device 101 may determine in operation 921 that a user location is home, company, vehicle, outdoors, or the like, or may determine, as a device state in operation 922, that the device is making a call, submerged, or located inside a pocket or a bag.

In operation 930, the electronic device 101 may configure the measurement profile on the basis of a data processing result. For example, the electronic device 101 may configure at least one among a measurement temperature profile 931, a measurement period profile 932, or an individual array operation profile 933 of the gas sensor by using the data processing result.

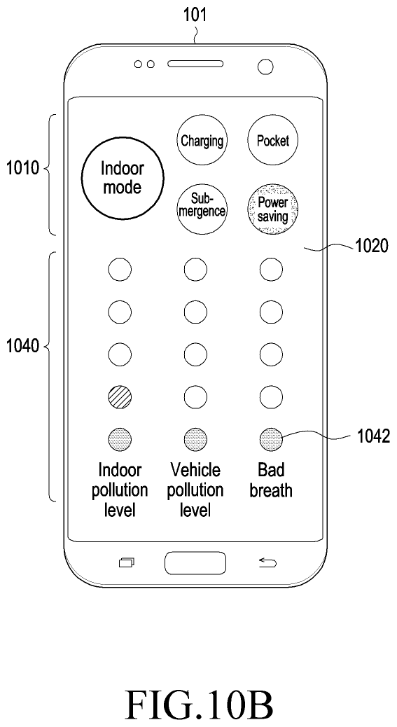

FIG. 10A illustrates conceptual diagrams for explaining an operation of performing output on a lock screen of the electronic device and an operation of performing output to another electronic device according to an embodiment.

In the disclosure, the electronic device 101 may display gas sensing numeric value information 1011. The electronic device 101 may output gas sensing data as the gas sensing numeric value information 1011 including a numeric value of a sensed gas amount. The electronic device 101 may output the gas sensing data as gas sensing evaluation information 1012 including a level of the sensed gas amount (e.g., an indoor pollution level, a vehicle pollution level, and bad breath). The gas sensing numeric value information 1011 may include numeric value information extracted from the gas sensing data. The gas sensing evaluation information 1012 may include evaluation information obtained by leveling the numeric value information. The gas sensing evaluation information 1012 may be acquired by comparing the numeric value of the gas amount and a threshold value.

The electronic device 101 may transmit a measurement result to another electronic device. For example, when the electronic device 101 transmits the gas sensing numeric value information 1011 to another electronic device 102 differing from the electronic device 101, the another electronic device 102 may output the gas sensing numeric value information 1011. In various embodiments of the disclosure, the other electronic device 102 may output the gas sensing numeric value information 1011 on a lock screen 1010. In various embodiments of the disclosure, the electronic device 101 may display the gas sensing numeric value information 1011 on a lock screen, as, for example, a configuration of the screen of the another electronic device 102 in FIG. 10A. In particular, the electronic device 101 may acquire, from the gas sensing data, a measured target gas name and gas sensing numeric value information including a concentration value of the target gas. The electronic device 101 may output the gas sensing numeric value information on the lock screen. The electronic device 101 may output the gas sensing numeric value information not only on a background screen but also on a screen of a currently executed application.

FIG. 10B illustrates conceptual diagrams for explaining a user interface of the electronic device according to an embodiment.

The electronic device 101 may output a gas measurement result to a user through a user interface. In particular, the electronic device 101 may output the gas sensing data and at least one of the ambient state information or the device state information by using the display as the user interface.

The electronic device 101 may output the gas sensing data in various schemes. The electronic device 101 may output the gas sensing data via sound such as a voice or an alert sound.

The electronic device 101 may visually output the gas sensing data to the user. For example, the electronic device 101 may gradually display a pollution level in a measurement result display area 1040. As the pollution level becomes worse, the electronic device 101 may differentiate the pollution level using different colors (e.g., green, blue, and red) and may show the user the pollution level using an icon or a light of a color corresponding to the pollution level. Further, as the pollution level becomes worse, the electronic device 101 may show the user the pollution level according to “good-moderate-bad” or “level 1-level 2-level 3-level 4-level 5”.

In FIG. 10B, an example of using the display as the user interface is illustrated. The electronic device 101 may display at least one of the ambient state information or the device state information in a state information display area 1030 of a screen 1020. The ambient state information may be displayed for each mode in the state information display area 1030. For example, a mode capable of displaying the ambient state information may include the call mode, the indoor mode, the vehicle mode, the outdoor mode, and the like. The device state information may be displayed using a term indicating operation and characteristics of the electronic device 101 in the state information display area 1030. For example, the term capable of indicating the operation and characteristics of the electronic device 101 may include charging, submergence, energy saving, and a state of being in a pocket with respect to the electronic device 101. The mode may be understood as a situation that the electronic device 101 is directly in. The call mode may include a situation in which the user is on the phone. The indoor mode may include a situation in which the electronic device 101 is located indoors, a vehicle mode may include a situation in which the electronic device 101 is in a vehicle, and the outdoor mode may include a situation in which the electronic device 101 is located in a place other than indoors. A basic mode may include a situation that does not belong any of the call mode, the indoor mode, the vehicle mode, and the outdoor mode.

The electronic device 101 may display gas sensing data in the measurement result display area 1040 of the screen 1020. The gas sensing data may be displayed as a numeric value. For example, the numeric value may be displayed together with a unit, such as parts per million (PPM) or percent (%). The gas sensing data may be displayed as an evaluation value. For example, an item for evaluation of the gas sensing data may include an indoor pollution level, a vehicle pollution level, and a bad breath level.

Because the electronic device 101 is located indoors, the indoor mode may be displayed in the state information display area 1030 of the screen 1020. When the electronic device 101 operates based on an energy saving, an energy saving indication may be displayed in the state information display area 1030 of the screen 1020. The gas sensing data may be converted into gas sensing evaluation information 1042 including an indoor pollution level, a vehicle pollution level, and a bad breath level, and may be displayed in the measurement result display area 1040 of the screen 1020. Because the electronic device 101 is located indoors, a response to the indoor pollution level may be displayed high.

In another embodiment, when the electronic device 101 located in a vehicle is in a charging operation, an indicator of “charging” in the information display area 1910 may be displayed. In this case, because the electronic device 101 is located in the vehicle, a response to the vehicle pollution may be displayed high.

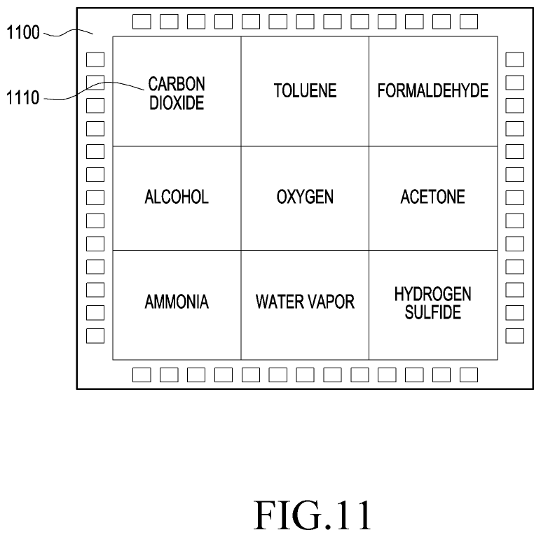

FIG. 11 illustrates an array gas sensor 1100 according to various embodiments of the disclosure. The array gas sensor 1100 may include multiple gas sensor cells 1110 capable of independently sensing the target gas. For example, the array gas sensor 1100 may be a type of an electronic nose (E-nose). Target gases, sensing temperatures, sensing periods, and types of gas sensors of the respective gas sensor cells 1110 may be independently determined, and the multiple gas sensor cells 1100 may be independently operated.

As an embodiment, the array gas sensor 1100 may include nine gas sensor cells 1110 in a 3×3 matrix form. Each of the nine gas sensor cells 1110 may sense carbon dioxide, toluene, formaldehyde, alcohol, oxygen, acetone, ammonia, water vapor, and hydrogen sulphide. The electronic device 101 may select the target gas by using ambient context information and device state information. In addition, the electronic device 101 may configure temperature according to a gas type for each of the gas sensor cells 1110. When the temperature of the target gas for each cell is achieved, a gas may be measured for each cell and, accordingly, a measurement time may be reduced compared to using one type of a sensor.

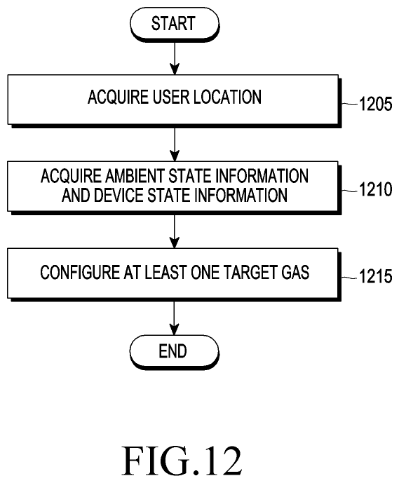

FIG. 12 illustrates a flow chart of gas measurement by the electronic device according to an embodiment.

In operation 1205, the electronic device 101 or the processor 210 may acquire location information of a user. For example, the electronic device 101 may determine whether the electronic device 101 is disposed outside or inside of a building or disposed inside a vehicle, on the basis of GPS coordinates, a signal received from a Wi-Fi communication device disposed indoors, and various schemes, such as paring with a Bluetooth communication device installed inside the vehicle.

In operation 1210, the electronic device 101 may acquire ambient state information and device state information. In operation 1215, the electronic device 101 may configure a target gas on the basis of at least one of the location information of the user, the ambient state information, or the device state information.

In various embodiments of the disclosure, the electronic device 101 may determine whether the user is on the phone, on the basis of at least one of the location information of the user, the ambient state information, or the device state information. When the user is on the phone, the electronic device 101 may configure a first target gas in a measurement profile. Here, the first target gas may include a gas required to be measured while the user is on the phone. For example, the first target gas may include carbon dioxide that the user on the phone breathes out.

In various embodiments of the disclosure, the electronic device 101 may determine that the electronic device 101 is located indoors (e.g., inside a building). When the electronic device 101 is located indoors, the electronic device 101 may configure a second target gas in the measurement profile. Here, the second target gas may include a gas required to be measured when the electronic device 101 is located indoors. For example, the second target gas may include oxygen that affects the comfort of a room.

In various embodiments of the disclosure, the electronic device 101 may determine that the electronic device 101 is located inside a vehicle. When the electronic device 101 is located inside the vehicle, the electronic device 101 may configure a third target gas in the measurement profile. The third target gas may include a gas required to be measured when the electronic device 101 is located inside the vehicle. For example, the third target gas may include hydrocarbon generated by combustion of fuel permeating the inside the vehicle.

In various embodiments of the disclosure, the electronic device 101 may configure, as the target gas, a basic gas in the measurement profile. The basic gas may include a gas required to be measured when the electronic device 101 is in a situation other than the described situation (a situation where the user is on the phone or the electronic device 101 is located indoors or in the vehicle).

In various embodiments of the disclosure, the electronic device 101 may configure a sensing temperature corresponding to at least one of the first target gas, the second target gas, the third target gas, and the basic gas. The electronic device 101 may configure a sensing period corresponding to at least one of the first target gas, the second target gas, the third target gas, and the basic gas. The electronic device 101 may sense at least one of the first target gas, the second target gas, the third target gas, and the basic gas according to the configured sensing temperature and sensing period.

In various embodiments of the disclosure, the electronic device 101 may determine two or more states. For example, the electronic device 101 may determine that the user is on the phone inside the vehicle. In this case, the electronic device 101 may configure multiple target gases or may sense multiple target gases sequentially or concurrently.

The electronic device 101 according to various embodiments of the disclosure may determine various states during a call, in addition to whether the call is made indoors or in a vehicle. For example, the electronic device 101 may determine whether the electronic device 101 is located in a bag. The electronic device 101 may determine that the electronic device 101 is located in a bag if it is determined that data sensed by the illumination sensor shows an environment in which light is blocked. In this case, the electronic device 101 may configure, as the target gas, a gas measured inside the bag, or may configure the measurement period to be relatively long. The electronic device 101 according to various embodiments of the disclosure may determine whether the electronic device 101 has been submerged. The electronic device 101 may determine whether submergence has occurred, on the basis of a measurement result of various submergence determination circuits included therein. When the electronic device 101 is determined to be submerged, the electronic device 101 may stop measurement.

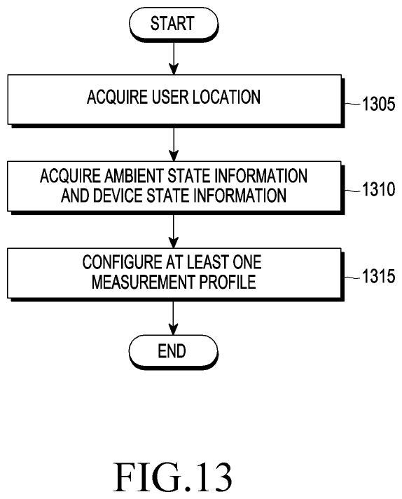

FIG. 13 illustrates a flow chart of gas measurement by the electronic device according to an embodiment.

In operation 1305, the electronic device 101 or the processor 210 may acquire location information of a user. In operation 1310, the electronic device 101 may acquire ambient state information and device state information. In operation 1315, the electronic device 101 may determine a measurement profile on the basis of at least one of the location information of the user, the ambient state information, or the device state information.

In various embodiments of the disclosure, the electronic device 101 or the processor 210 may determine whether the user is on the phone, on the basis of at least one of the ambient state information or the device state information. When the user is on the phone, the electronic device 101 may configure the measurement profile to the call mode. The configuration of the measurement profile may include at least one of selection of a target gas to be measured, a sensing temperature configuration, or a sensing period configuration. For example, configuration to the call mode for the measurement profile may include configuring the measurement profile by configuration of the target gas required to be measured when the user is on the phone, and by configuration of a sensing temperature and a sensing period. For example, when the measurement profile is configured to the call mode, the electronic device 101 may configure the target gas to be carbon dioxide, may configure the sensing temperature to be 120° C. (within ±10° C.), and may configure the sensing period to be 30 times/sec.

In various embodiments of the disclosure, the electronic device 101 may determine that the electronic device 101 is located indoors. When the electronic device 101 is located indoors, the electronic device 101 may configure the measurement profile to be the indoor mode. The configuration to the indoor mode of the measurement profile may include configurations of a target gas required to be measured, a sensing temperature, and a sensing period when the electronic device 101 is located indoors.

For example, when at least one of the ambient state information and the device state information indicates the indoor mode, the electronic device 101 may configure the target gas to be carbon dioxide, carbon monoxide, and oxygen and may configure the sensing temperature to be 120° C. (within a range of ±10° C.) and a sensing period to be 40 times/sec.

In various embodiments of the disclosure, the electronic device 101 may determine that the electric 101 is located inside a vehicle. When the electric 101 is located inside the vehicle, the electronic device 101 may configure the measurement profile to be the vehicle mode. The configuration of the measurement profile to the vehicle mode may include configuring the measurement profile by configurations of a target gas required to be measured, and by configuration of a sensing temperature and a sensing period, when the electronic device 101 is located inside the vehicle. For example, the electronic device 101 may configure the target gas to be carbon dioxide, carbon monoxide, hydrocarbons and oxygen, and may configure the sensing temperature to be 450° C. (within a range of ±50° C.) and the sensing period to be 60 times/sec.

In various embodiments of the disclosure, the electronic device 101 may configure the measurement profile to the basic mode. The configuration to the basic mode of the measurement profile may include configurations of a target gas required to be measured, a sensing temperature, and a sensing period when the electronic device 101 does not correspond to the modes (a situation where a user is on the phone or the electronic device 101 is located indoors or inside a vehicle). For example, the electronic device 101 may configure the target gas to be carbon dioxide and oxygen, and may configure the sensing temperature to be 120° C. (within a range of ±10° C.) and the sensing period to be 10 times/sec.

On the other hand, although not shown, for example, when the measurement profile is determined to have the outdoor mode, the electronic device 101 may configure the target gas to be carbon dioxide, hydrocarbons, sulfurous acid gas, and nitrogen oxide, and may configure the sensing temperature to be 250° C. (within a range of ±50° C.) and the sensing period to be 10 times/sec.

The electronic device 101 may sense the target gas according to the measurement profile configured according to one of the call mode, the indoor mode, the vehicle mode, or the basic mode.

In various embodiments of the disclosure, the electronic device 101 may select multiple modes. For example, the electronic device 101 may determine that a user is on the phone inside a vehicle, and may select measurement profiles corresponding to the vehicle mode and the call mode, respectively. The electronic device 101 may sense multiple measurement profiles sequentially or concurrently, and sense a target gas. When there is a common target gas among the multiple measurement profiles, the electronic device 101 may sense the common target gas once, instead of sensing the target gas in a duplicate manner. The electronic device 101 may display information of multiple target gases.

[Table 1] is an example of a measurement profile for each mode according to various embodiments of the disclosure.

| | | | | |
|---|---|---|---|---|
| Indoor | Carbon dioxide | 350° C. (±10) | 40 times/sec | Battery |
| mode | (CO₂) | | | chemical gas |
| | Carbon | | | sensor |
| | monoxide (CO) | | | |
| | Oxygen (O₂) | | | |
| Vehicle | Carbon dioxide | 450° C. (±50) | 60 times/sec | Semiconductor |
| mode | (CO₂) | | | gas sensor |
| | Carbon | | | |
| | monoxide (CO) | | | |
| | Hydrocarbon | | | |
| | (CₓHₓ) | | | |
| | Oxygen (O₂) | | | |
| Outdoor | Carbon dioxide | 250° C. (±50) | 10 times/sec | Array gas |
| mode | (CO₂) | | | sensor |
| | Hydrocarbon | | | |
| | (CₓHₓ) | | | |
| | Sulfurous acid | | | |
| | gas (SO₂) | | | |
| | Nitrogen oxide | | | |
| | (NOₓ) | | | |

In various embodiments of the disclosure, the electronic device 101 may determine that the electronic device 101 is located outdoors. In this case, the electronic device 101 may determine the target gas or the measurement profile on the basis of a pollution level of a corresponding region or Internet weather forecast, gas measurement information of another user, map information for updating gas information in real time, and the like.

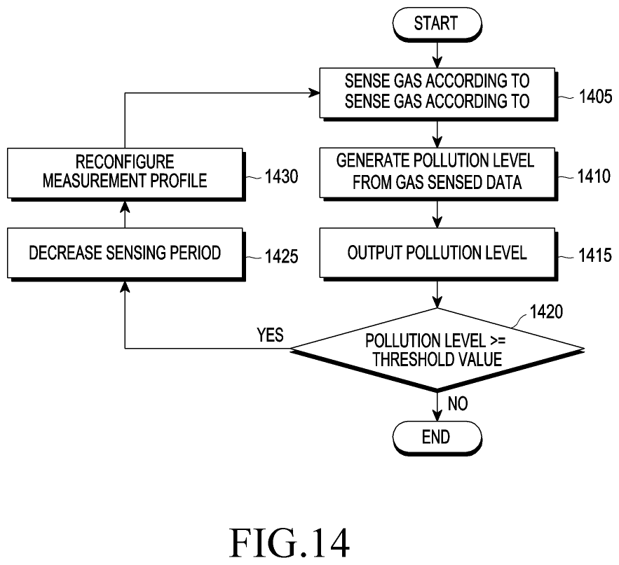

FIG. 14 illustrates a flow chart of repetitive sensing by the electronic device according to an embodiment.

The electronic device 101 may repeat gas sensing for a target gas according to a measurement result.

In operation 1405, the electronic device 101 or the processor 210 may sense a gas according to the measurement profile. In operation 1410, the electronic device 101 may process the gas sensing data so as to acquire a pollution level. In operation 1415, the electronic device 101 may output the pollution level. In operation 1420, the electronic device 101 may determine whether to repeat sensing. For example, the electronic device 101 may determine whether the pollution level is greater than or equal to a threshold value. When the pollution level is greater than or equal to the threshold value, the electronic device 101 may sense the target gas again according to the measurement profile.

When air pollution is serious, it is advantageous to increase the number of sensing times so as to sense the target gas more frequently. In an opposite case, it is advantageous in terms of power saving to decrease a sensing period, so as to sense the target gas intermittently. Therefore, in operation 1425, when the pollution level is greater than or equal to the threshold value, the electronic device may decrease the sensing period, that is to increase a measurement frequency.

In operation 1430, the electronic device 101 may reconfigure the measurement profile according to the decreased sensing period. Later, the already configured target gas may be sensed again according to the reconfigured measurement profile.

When the pollution level is equal to or greater than the threshold value, the electronic device 101 according to various embodiments of the disclosure may or optionally may not increase the sensing period and reconfigure the measurement profile.

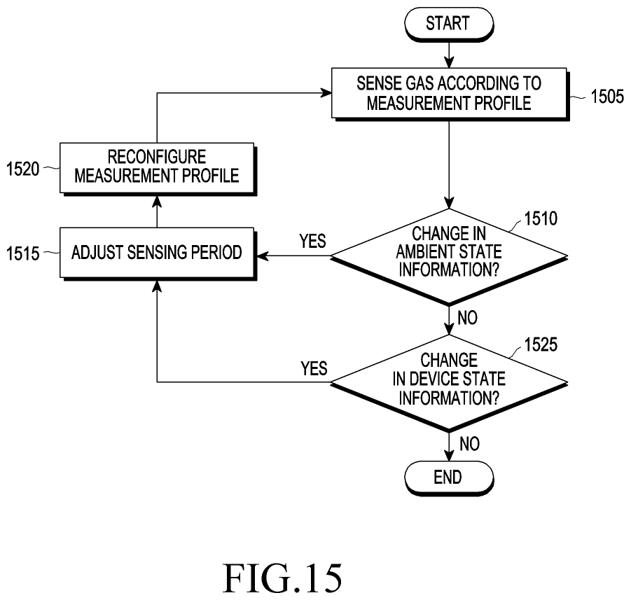

FIG. 15 illustrates a flow chart of an operation of adjusting a sensing period on the basis of a change in ambient state information and device state information of the electronic device according to an embodiment.

In operation 1505, the electronic device 101 or the processor 210 may sense a gas according to the measurement profile. In operation 1510, the electronic device 101 may determine whether the ambient state information has changed. The change in the ambient state information may include a change in a user's state and a change in an environment surrounding the electronic device 101. For example, the change in the ambient state information may include a case where the electronic device 101 was located indoors and then moves to the outside or a vehicle.

When the ambient state information is changed, the electronic device 101 may adjust, in operation 1515, a sensing period having been already configured in the measurement profile. In operation 1520, the electronic device 101 may reconfigure the measurement profile according to the adjusted sensing period. Later, the electronic device 101 may re-sense the already configured target gas, according to the reconfigured measurement profile.

When the device state information is not changed, the electronic device 101 may determine in operation 1525 whether the device state information is changed. The change in the device state information may include a change in operation or characteristics of the electronic device 101. For example, the change in the device state information may include a case where the electronic device 101 in a charging progress cannot receive power or a case where the electronic device 101 in a power saving mode receives power for charging.

When the device state information is changed, the electronic device 101 may adjust, in operation 1515, the sensing period configured in the measurement profile. In operation 1520, the electronic device 101 may reconfigure the measurement profile according to the adjusted sensing period. Later, the electronic device 101 may re-sense the already configured target gas, according to the reconfigured measurement profile.

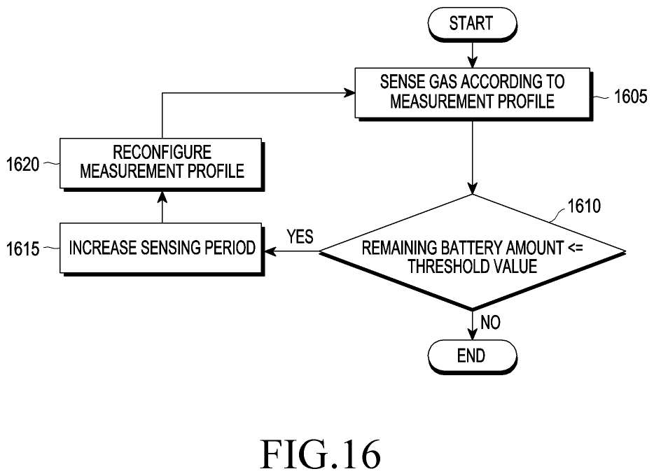

FIG. 16 illustrates a flow chart of sensing period adjustment on the basis of a remaining amount of battery power of the electronic device according to an embodiment.

Referring to FIG. 16, the electronic device 101 or the processor 210 may adjust a sensing period according to the remaining battery amount. In operation 1605, the electronic device 101 may sense a gas according to a measurement profile. In operation 1610, the electronic device 101 may determine whether to adjust the sensing period. For example, the electronic device 101 may determine whether the remaining battery amount is less than or equal to a threshold value.

If the battery power is insufficient, it is advantageous in terms of power saving to decrease a sensing frequency so as to sense the gas intermittently. Therefore, in operation 1615, when the remaining battery amount is less than or equal to the threshold value, the electronic device 101 may increase the sensing period, that is to decrease a measurement frequency.

In operation 1620, the electronic device 101 may reconfigure the sensing period in the measurement profile according to the increased sensing period. Later, the electronic device 101 may re-sense the already configured target gas, according to the reconfigured measurement profile.

The electronic device 101 according to various embodiments of the disclosure may determine whether the electronic device 101 is placed inside a bag. The electronic device 101 may determine that the electronic device 101 is located in a bag if it is determined that data sensed by the illumination sensor shows an environment in which light is blocked. In this case, the electronic device 101 may configure a measurement period to be relatively long. The electronic device 101 according to various embodiments of the disclosure may determine whether the electronic device 101 has been submerged. The electronic device 101 may determine whether submergence has occurred, on the basis of a measurement result of various submergence determination circuits included therein. When the electronic device 101 is determined to be submerged, the electronic device 101 may stop measurement. The electronic device 101 according to various embodiments of the disclosure may determine whether the remaining battery amount is less than a preconfigured threshold value. When it is determined that the remaining battery amount is less than the threshold value, the electronic device 101 may configure the measurement period to be relatively long.

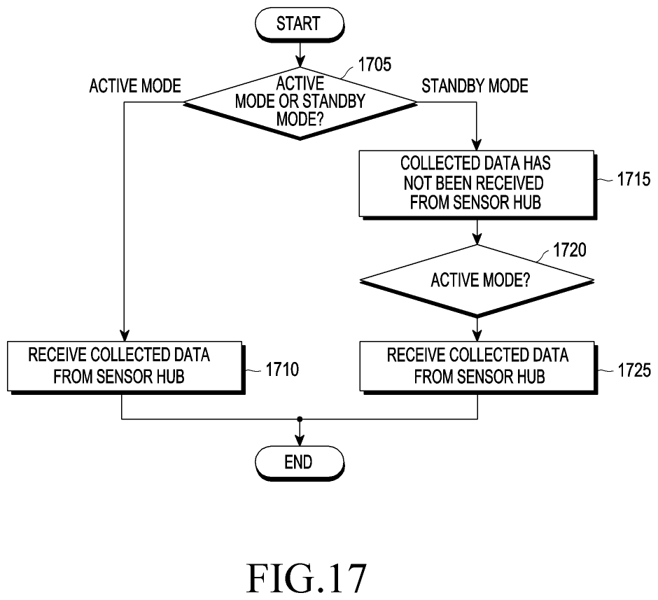

FIG. 17 illustrates a flow chart of a processor operation in a standby mode according to an embodiment.

After the sensor hub collects data through sensors for data collection, the sensor hub may temporarily store the collected data, without transmitting the collected data to the processor 210. When the sensor hub receives a data transmission request of the processor 210, the sensor hub may transmit the collected data to the processor 210. For example, when the processor 210 has not performed an operation for a while, the sensor hub may store the collected data.

Referring to FIG. 17, a flow of operations of receiving collected data by the processor 210 having a standby mode is illustrated. In the standby mode, the processor 210 may pause unnecessary operations for a while in order to minimize power consumption. In an active mode, which is a concept corresponding to the standby mode, the processor 210 may perform all conventional operations without pausing. Therefore, the processor 210 may perform data reception from the sensor hub in a different scheme depending on the standby mode or the active mode.

In operation 1705, the processor 210 may determine whether a state of the processor 210 corresponds to one of the active mode or the standby mode. When the state of the processor 210 corresponds to the active mode, the processor 210 may receive collected data from the sensor module 240 in operation 1710.

When the state of the processor 210 corresponds to the standby mode, the processor 210 does not receive all the collected data in operation 1715. In operation 1720, the processor 210 may re-determine whether the state of the processor 210 corresponds to the active mode or the standby mode.

When the state of the processor 210 corresponds to the active mode, the processor 210 may request data transmission from the sensor hub and may receive the collected data from the sensor hub, in operation 1725. Until the request arrives to the sensor hub, the collected data may be temporarily stored in the sensor hub. When the state of the processor 210 corresponds to the standby mode, the processor 210 may not still receive any collected data.

A method for measuring a gas by an electronic device including a sensor module according to various embodiments of the disclosure may include: acquiring at least one of ambient state information indicating information relating to the outside of the electronic device or device state information indicating information relating to the inside of the electronic device; acquiring a measurement profile including a target gas to be measured and information relating to a sensing period of the target gas, on the basis of at least one of the ambient state information or the device state information; and sensing the target gas through the sensor module according to the measurement profile.

Acquiring of the measurement profile according to various embodiments of the disclosure may include: configuring the target gas; configuring a sensing temperature of the target gas; and configuring the sensing period of the target gas.

Acquiring of the measurement profile according to various embodiments of the disclosure may include, when the sensor module includes sensor cells that sense multiple gases, configuring the target gas, the sensing temperature, and the sensing period for each of the sensor cells.

Acquiring of the measurement profile according to various embodiments of the disclosure may include configuring multiple target gases and configuring sensing temperatures and sensing periods for the respective multiple target gases.

Acquiring of the measurement profile according to various embodiments of the disclosure may include configuring a sensing order or iterative sensing of the multiple target gases.

The method according to various embodiments of the disclosure may further include adjusting the sensing period according to a measurement result of the target gas, and acquiring of the measurement profile may include reconfiguring the sensing period to be the adjusted sensing period.

The method according to various embodiments of the disclosure may further include adjusting the sensing period according to a remaining battery amount, submergence, sound data transmission or reception of the electronic device, or an aspect in which the electronic device is kept, and acquiring of the measurement profile may include reconfiguring the sensing period to be the adjusted sensing period.

The method according to various embodiments of the disclosure may further include adjusting the sensing period according to a place where the electronic device is located, and acquiring of the measurement profile may include reconfiguring the sensing period to be the adjusted sensing period.

The term “module” used in the present document may refer to, for example, a unit including one or a combination of two or more among hardware, software, and firmware. The “module” may be interchangeably used with terms, such as unit, logic, logical block, component, circuit, or the like. The “module” may be a minimum unit of an integrated component element or a part thereof. The “module” may be a minimum unit for performing one or more functions or a part thereof. The “module” may be mechanically or electronically implemented. For example, the “module” according to the disclosure may include at least one of an Application-Specific Integrated Circuit (ASIC) chip, a Field-Programmable Gate Arrays (FPGA), and a programmable-logic device for performing operations which has been known or are to be developed hereinafter.

According to various embodiments, at least some of the devices (for example, modules or functions thereof) or the method (for example, operations) according to the disclosure may be implemented by a command stored in a computer-readable storage medium in a programming module form. When the command is executed by one or more processors (for example, the processor 120), the one or more processors may execute a function corresponding to the command. The computer-readable storage medium may, for example, be the memory 130.

The computer readable recoding medium may include a hard disk, a floppy disk, magnetic media (e.g., a magnetic tape), optical media (e.g., a Compact Disc Read Only Memory (CD-ROM) and a Digital Versatile Disc (DVD)), magneto-optical media (e.g., a floptical disk), a hardware device (e.g., a Read Only Memory (ROM), a Random Access Memory (RAM), a flash memory), and the like. In addition, the program instructions may include high class language codes, which can be executed in a computer by using an interpreter, as well as machine codes made by a compiler. The aforementioned hardware device may be configured to operate as one or more software modules in order to perform the operation of the disclosure, and vice versa.

According to various embodiments of the disclosure, a recording medium stores instructions, the instructions configured to cause, when executed by at least one processor, the at least one processor to perform at least one operation in a method of measuring a gas by an electronic device including a sensor module, wherein the method includes: acquiring at least one of ambient state information indicating information relating to the outside of the electronic device or device state information indicating information relating to the inside of the electronic device; acquiring a measurement profile including a target gas to be measured and information relating to a sensing period of the target gas, on the basis of at least one of the ambient state information or the device state information; and sensing the target gas through the sensor module according to the measurement profile.

Various embodiments disclosed herein are provided merely to easily describe technical details of the disclosure and to help the understanding of the disclosure, and are not intended to limit the scope of the disclosure. Therefore, it should be construed that all modifications and changes or modified and changed forms based on the technical idea of the disclosure fall within the scope of the disclosure.

1. An electronic device comprising:
a sensor module; and
a processor configured to: acquire at least one of ambient state information indicating information relating to outside of the electronic device or device state information indicating information relating to inside of the electronic device; acquire a measurement profile comprising a target gas to be measured and information relating to a sensing period of the target gas, on the basis of the at least one of the ambient state information or the device state information; and sense the target gas through the sensor module according to the measurement profile.

2. The electronic device of claim 1, wherein the processor is configured to configure, in the measurement profile, the target gas, a sensing temperature of the target gas, and the sensing period of the target gas.

3. The electronic device of claim 2, wherein, when the sensor module comprises sensor cells configured to sense multiple gases, the processor configures the target gas, the sensing temperature, and the sensing period for each of the sensor cells in the measurement profile.

4. The electronic device of claim 2, wherein the processor is configured to configure, in the measurement profile, multiple target gases, and configures sensing temperatures and sensing periods for the respective multiple target gases.

5. The electronic device of claim 4, wherein the processor is configured to configure a sensing order or iterative sensing of the multiple target gases in the measurement profile.

6. The electronic device of claim 2, wherein the processor is configured to adjust the sensing period according to a measurement result of the target gas, and reconfigure the sensing period to be the adjusted sensing period.

7. The electronic device of claim 2, wherein the processor is configured to adjust the sensing period according to a change in the ambient state information and a change in the device state information, and reconfigure the sensing period to be the adjusted sensing period.

8. The electronic device of claim 2, wherein the processor is configured to adjust the sensing period according to a remaining battery amount, submergence, sound data transmission or reception of the electronic device, or an aspect in which the electronic device is kept, and reconfigure the sensing period to be the adjusted sensing period.

9. The electronic device of claim 2, wherein the processor is configured to adjust the sensing period according to a place where the electronic device is located, and reconfigure the sensing period to be the adjusted sensing period.

10. The electronic device of claim 2, further comprising:
a display; and a communication module,
wherein the processor is configured to output a measurement result of the target gas on the display or transmit the measurement result to another electronic device through the communication module.

11. The electronic device of claim 2, wherein the processor is configured to determine the sensing temperature based on a type of the target gas to be measured by the sensor module.

12. A method of measuring a gas by an electronic device comprising a sensor module, the method comprising:
acquiring at least one of ambient state information indicating information relating to the outside of the electronic device or device state information indicating information relating to the inside of the electronic device;
acquiring a measurement profile comprising a target gas to be measured and information relating to a sensing period of the target gas, based on the at least one of the ambient state information or the device state information; and
sensing the target gas through the sensor module according to the measurement profile.

13. The method of claim 12, wherein the acquiring of the measurement profile comprises:
configuring the target gas;
configuring a sensing temperature of the target gas; and
configuring the sensing period of the target gas.

14. The method of claim 13, wherein the acquiring of the measurement profile comprises, when the sensor module comprises sensor cells configured to sense multiple gases, configuring the target gas, the sensing temperature, and the sensing period for each of the sensor cells.

15. The method of claim 13, wherein the acquiring of the measurement profile comprises configuring multiple target gases, and configuring sensing temperatures and sensing periods for the respective target gases.

16. The method of claim 15, wherein the acquiring of the measurement profile comprises configuring a sensing order or iterative sensing of the multiple target gases in the measurement profile.

17. The method of claim 13, further comprising adjusting the sensing period according to a measurement result of the target gas, and
wherein the acquiring of the measurement profile comprises reconfiguring the sensing period to be the adjusted sensing period.

18. The method of claim 13, further comprising adjusting the sensing period according to a remaining battery amount, submergence, sound data transmission or reception of the electronic device, or an aspect in which the electronic device is kept, and
wherein the acquiring of the measurement profile comprises reconfiguring the sensing period to be the adjusted sensing period.

19. The method of claim 13, further comprising adjusting the sensing period according to a place where the electronic device is located, and
wherein the acquiring of the measurement profile comprises reconfiguring the sensing period to be the adjusted sensing period.

20. A non-transitory, computer-readable storage medium storing instructions for performing the method according to claim 12.
